(19) United States
(12) Patent Application Publication
(10) Pub. No.: US 2013/0149734 A1
(43) Pub. Date: Jun. 13, 2013

(54) Multi-photon Tissue Imaging

(71) Applicants: David Ammar, Denver, CO (US); Malik Kahook, Denver, CO (US); Tim Lei, Thornton, CO (US); Emily Gibson, Boulder, CO (US); Omid Masihzadeh, Lakewood, CO (US); Naresh Mandava, Denver, CO (US)

(72) Inventors: David Ammar, Denver, CO (US); Malik Kahook, Denver, CO (US); Tim Lei, Thornton, CO (US); Emily Gibson, Boulder, CO (US); Omid Masihzadeh, Lakewood, CO (US); Naresh Mandava, Denver, CO (US)

(73) Assignee: The Regents of the University of Colorado, a body corporate, Denver, CO (US)

(21) Appl. No.: 13/700,682

(22) PCT Filed: May 31, 2011
(86) PCT No.: PCT/US11/38655

§ 371 Date: Feb. 13, 2013

Related U.S. Application Data
(60) Provisional application No. 61/349,237, filed on May 28, 2010.
(60) Provisional application No. 61/361,747, filed on Jul. 6, 2010.

(51) Int. Cl. A61B 3/00; G01N 21/65; G01N 21/64

(52) U.S. Cl. CPC A61B 3/0008; G01N 21/6486; G01N 21/64; G01N 21/65
USPC 435/29; 600/476; 250/458.1; 356/73; 250/459.1

(57) ABSTRACT

A multimodal method for imaging tissue comprising: aligning an excitation light source with at least a portion of the tissue; selecting at least two modalities of image acquisition; imaging the tissue portion with each of the modalities of image acquisition; and constructing a dual mode image using images from each of the modalities of image acquisition. A multimodal system for imaging tissue comprising: an excitation light source or light sources; an optical and alignment system for directing the excitation beam or beams to a sample and receiving an emission beam from the sample; at least one detector for receiving the emission beam from the sample; and a spectral filtering or dispersing device for providing at least two imaging modalities at the at least one detector; and a processor for analyzing the detected emission beam and constructing a dual mode image using images from each of the modalities of image acquisition.

### CROSS-REFERENCE TO RELATED APPLICATIONS

This application claims the benefit of U.S. provisional application No. 61/349,247, filed 28 May 2010 and U.S. provisional application No. 61,361,747 filed 6 Jul. 2010, each of which is hereby incorporated by reference as though fully set forth herein.

### BACKGROUND

a. Field of the Invention

The instant invention relates to imaging tissue, more specifically the instant invention relates to imaging tissue using multi-photon microscopy (MPM).

b. Background

Imaging modalities such as digital photography and ultrasound have become integral in the clinical and surgical practice of ophthalmology over the past few decades. More recently, diode laser based imaging devices such as GDx, Heidelberg Retinal Tomography (HRT), and optical coherence tomography (OCT) have been used in the examination and early diagnosis of disease ranging from macular degeneration to glaucoma. Despite these advances, the aforementioned imaging devices are restricted in their ability to image tissue structure while being unable to elucidate tissue function. This limitation becomes even more important when noting that the structural normative databases used to delineate abnormal from normal tissue have inherent limitations. Physiologic differences from patient to patient as well as coexisting conditions, such as thinning of the retinal nerve fiber layer (RNFL) in high myopia, may alter the structure of tissues but often do not alter actual visual function.

Multi-photon microscopy (MPM) has found increasing use in laboratory based biomedical imaging due to its sub-cellular resolution along with the ability to obtain structural and functional information. These properties make MPM unique compared to other imaging modalities such as ultrasound, magnetic resonance imaging (MRI), or X-ray/computed tomography (CT) imaging. However, to achieve these benefits, there is a drawback in the limited tissue penetration depth as well as the ability to image highly scattering tissues such as sclera.

### SUMMARY

A system, method, and apparatus for imaging tissue (e.g., eye tissue) using multi-photon microscopy. In one embodiment, for example, multi-photon microscopy may be used to image living tissue in an intact eye. The imaging, for example, may be performed in vivo or ex vivo.

In one particular implementation, for example, the imaging is performed by scanning for multiple axis image detection. An alignment mechanism is used to locate a region to be imaged. The alignment mechanism, for example, may include incorporated spectral optical coherence tomography or confocal reflectance imaging capabilities.

The imaging may be performed without labels using multimodal image acquisition including at least two of the following imaging techniques: two photon excitation fluorescence/autofluorescence, fluorescence/autofluorescence lifetime, second harmonic generation, third harmonic generation, coherent anti-Stokes Raman scattering (CARS) spectroscopy, broadband or multiplexCARS (B-CARS or M-CARS), stimulated Raman scattering (SRS), stimulated emission, nonlinear absorption, micro-Raman spectroscopy, and the like.

In some implementations where the imaging target is located within the intact eye, a long working distance objective or an imaging lens is used to access the target (e.g., a trabecular meshwork region of the intact eye).

In one particular embodiment, for example, a new diagnostic paradigm for diagnosing eye diseases, such as glaucoma, in vivo using multi photon microscopy. While clinical light-imaging techniques currently in use cannot image TM cells within living tissue, multi-photon imaging technology can provide greater penetration depth and spatial resolution. The use of multi-photon microscopy in a clinical environment provides many practical advantages to techniques that use visible light or ultrasound. The sensitivity of retinal chromophores to the near infra-red laser (800 nm) is low, resulting in greater patient comfort. The laser pulses have high peak power, but due to the extremely short pulse duration (˜100 femtoseconds) have a low average power. This, combined with tight focusing, leads to efficient two-photon excitation with low power absorption and thermal exposure to the tissue. Finally, the resolution of the multi-photon microscope has the potential to analyze living tissue with histological accuracy without actually taking a biopsy sample. Living skin has been imaged by two photon microscopy to a depth of 350 microns by visualizing the autofluorescence of the skin's extracellular matrix and melanin. The experimentally measured resolution was determined to be 0.5-1 microns lateral by 3-5 microns axial, which is on par with typical resolution of a 5 micron thick histological section.

The foregoing and other aspects, features, details, utilities, and advantages of the present invention will be apparent from reading the following description and claims, and from reviewing the accompanying drawings.

### BRIEF DESCRIPTION OF THE DRAWINGS

FIGS. 1A-1D show schematics of example processes that result from nonlinear multi-photon interactions with a molecule.

FIGS. 1E-1F are energy diagrams of the Two-Photon Autofluorescence (TPAF) (FIG. 1E) and Coherent Anti-Stokes Raman Scattering (CARS) (FIG. 1F).

FIGS. 2A and 2B illustrate a time-domain FLIM process and a frequency domain FLIM process.

FIGS. 3A and 3B show schematics of descanned (FIG. 3A) and non-descanned (FIG. 3B) example embodiment configurations for performing multi-photon (MP) imaging.

FIG. 4 shows a diagram of an eye highlighting example regions for multi-photon microscopy (MPM) imaging.

FIG. 5 illustrates a general schematic of the optical configuration of a multi-photon imaging system.

FIG. 6 shows a vascular bed of a human retina imaged by second harmonic generation (SHG).

FIG. 7 shows a schematic view of the trabecular meshwork (TM) region of an eye.

FIG. 8A shows an example two-photon autofluorescence image of the TM and scleral strip.

FIG. 8B shows an example second harmonic generation image of collagen within the trabecular meshwork of an eye.

FIGS. 9A and 9B show a three dimensional reconstruction of images of the trabecular meshwork region by second harmonic generation imaging showing the front side (near sclera) and backside.

FIG. 10 shows an example standard histological section of the TM region of an eye.

FIG. 11 shows an example second harmonic generation and Hoechst fluorescence of flat mounted human trabecular meshwork eye tissue.

FIG. 12 shows an example three-dimensional reconstruction of a trans-scleral imaging using second harmonic generation and Hoechst fluorescence in human trabecular meshwork eye tissue.

FIGS. 13A-13C show example second harmonic generation (SHG) and two-photon autofluorescence (TPAF) images of the trabecular meshwork region of a human eye.

FIG. 14 shows an example CARS/TPAF multi-photon microscopy platform.

FIG. 15A shows example CARS/TPAF images are taken along a trabecular meshwork region.

FIG. 15B displays example CARS and TPAF channels in an image.

FIG. 15C shows the TPAF channel of FIG. 15B in isolation from the CARS channel.

FIGS. 17A-17D show example autofluorescence lifetime changes of TM epithelial cells upon addition of the preservative BAK.

FIGS. 17A, 17B, and 17C illustrate example routes for introducing an excitation beam into an intact eye.

FIG. 18 shows an example process for imaging a portion of an intact eye.

FIG. 19 illustrates an exemplary system useful in implementations of the described technology.

### DETAILED DESCRIPTION

Systems, methods, and apparatuses for imaging tissue, such as for imaging eye tissue in vivo (e.g., an intact eye) or ex vivo are provided. Although examples of imaging eye tissue are described in detail, various imaging modalities have application for imaging tissue outside of the eye. In addition, imaging of the trabecular meshwork in the anterior chamber of an eye are discussed merely as an example. Other tissues within the eye, such as cornea, conjunctiva, Schlemm's canal, collector channels, sclera, ciliary body, iris, lens, retina, choroid, optic nerve, vitreous, aqueous humor, blood vessels as well as tissues surrounding these structures, may also be imaged in vivo or ex vivo. The imaging of eye tissue may be performed for diagnostic purposes, during a surgical procedure in which the power of the same imaging laser may be increased for the surgical procedure or in which a separate laser or other surgical implement may be used in addition to the imaging laser source, and/or to monitor drug delivery.

In one particular implementation, tissue imaging may be performed in vivo without labels using multi-photon microscopy (MPM) techniques.

Traditional fluorescence microscopy (epifluorescence or confocal) is based on linear absorption processes: a single photon excites a fluorophore resulting in the emission of a photon with a longer wavelength. When using excitation light sources in the visible range, these events are confined to within 100 microns of the surface of the tissue due to light scattering.

In contrast, multi-photon microscopy is based on non-linear processes that involve multiple photons interacting with molecules in the sample. Since the probability of simultaneous interactions with two (or more) photons is extremely low (cross-sections on order of 10⁻⁵⁰ cm⁴ s or 1 GM), the process occurs when there is high photon flux (such as on the order of 10⁶-10⁸ W/cm²). This is typically achieved using a pulsed near-infrared laser with a pulse duration on order of ˜100 femtoseconds focused with a high numerical aperture objective. As a result, MPM offers intrinsic axial cross sectioning because the process only occurs at the focus of the microscope objective where the laser intensity is greatest. MPM imaging offers equivalent resolution as confocal microscopy (˜200 nm lateral and ˜1.0 micron axial) but does not require the use of a pinhole. An additional advantage of using a near-infrared laser source is deeper tissue penetration due to reduced light scattering with longer wavelengths of light. MPM can provide contrast without exogenous dye labeling and is a completely non-invasive technique.

Multi-photon microscopy includes the following imaging modalities: two photon excitation fluorescence (TPEF) or autofluorescence (TPAF), fluorescence lifetime imaging (FLIM), second harmonic generation (SHG), third harmonic generation (THG), coherent anti-stokes Raman scattering (CARS) spectroscopy, broadband or multiplex CARS (B-CARS or M-CARS), stimulated Raman scattering (SRS), stimulated emission, nonlinear absorption, micro-Raman spectroscopy and the like. Various embodiments provide both structural and functional imaging of the tissue that may allow a physician to make more informed decisions on surgery or course of treatment.

In two-photon excitation fluorescence (TPEF) imaging, intense short-pulsed near infrared light is focused into a small volume, thereby increasing the probability of two photons arriving ‘simultaneously’ at a target molecule. The combined energy is absorbed by the target molecule and released as a single photon. Imaging biological molecules (such as NAD(P)H, FAD, elastin, melanin, and lipofuscin) in this manner is often referred to as two-photon autofluorescence (TPAF), since the fluorescence results from the intrinsic properties of these molecules. Another two-photon process is second harmonic generation (SHG). In this case, two photons are simultaneously ‘scattered’ by a highly ordered asymmetric macromolecule (like collagen fibril), resulting in a single photon with a precise wavelength of half the excitation wavelength, that is distinct from any generated autofluorescence (AF). Due to the narrow SHG spectral peak and difference in wavelength, AF and SHG signals can be separated using spectral filtering and simultaneously detected.

CARS is a multi-photon imaging technique that is fundamentally different from both TPEF/TPAF and SHG. CARS is a nonlinear version of Raman spectroscopy. In the Raman process, a narrow band laser illuminates the sample and a portion of the incident photons are scattered by interactions with molecular vibrations, resulting in a shift to higher (anti-Stokes) or lower frequency (Stokes) photons. The signal intensity is very weak because of the extremely low scattering cross-section (˜10⁻³⁰ cm²/molecule) as opposed to the absorption cross-section of a typical fluorophore (˜10⁻¹⁵ cm²/molecule).

In contrast to traditional Raman spectroscopy, CARS is a nonlinear optical process that selectively and coherently excites vibrational resonances of biomolecules to rapidly obtain the Raman (vibrational) spectrum. Compared to traditional Raman scattering, the CARS process increases the detection sensitivity by up to 10⁷ to allow rapid data acquisition. With the associated decrease in measurement times, CARS can be applied in biomedical microscopy to image live cells at video rates without extrinsic fluorescence dye labeling. In the CARS process, two photons (pump and Stokes) excite a specific vibrational resonance coherently. A third photon (probe) subsequently measures the density of the vibrational resonance. The number of emitted anti-Stokes photons that are energy shifted by that vibrational mode is proportional to the square of the density of the vibrational oscillators, thus the molecular concentration. A traditional CARS setup uses two synchronized picosecond lasers or a single picosecond laser with an optical parametric amplifier to generate the two laser beams with different frequencies matched to one particular vibrational resonance. By tuning the laser frequency difference to a particular vibrational mode, for example 2850 cm⁻¹ of the CH₂ stretch for lipids, chemical-specific imaging can be achieved all without use of fluorescent dyes or other labeling techniques.

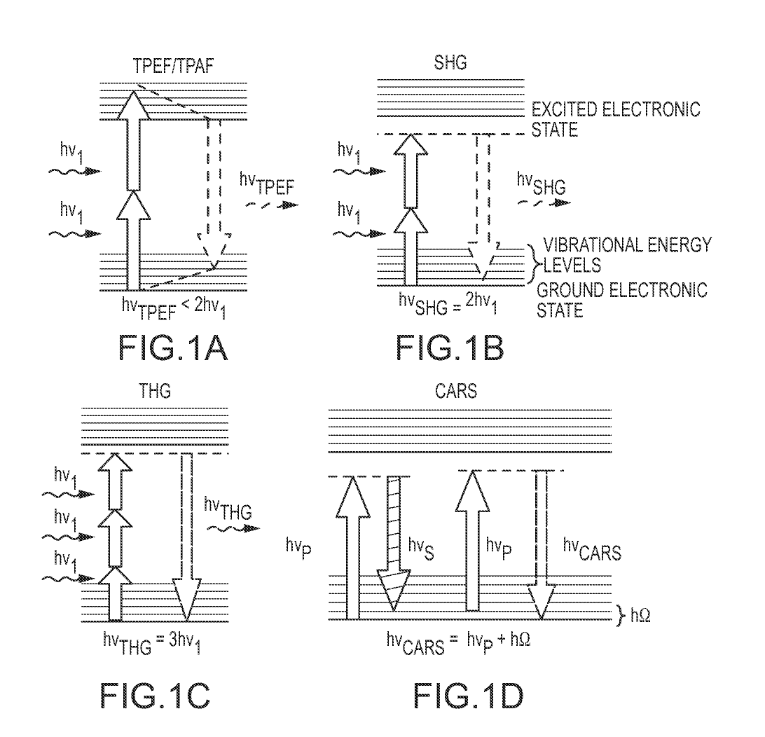

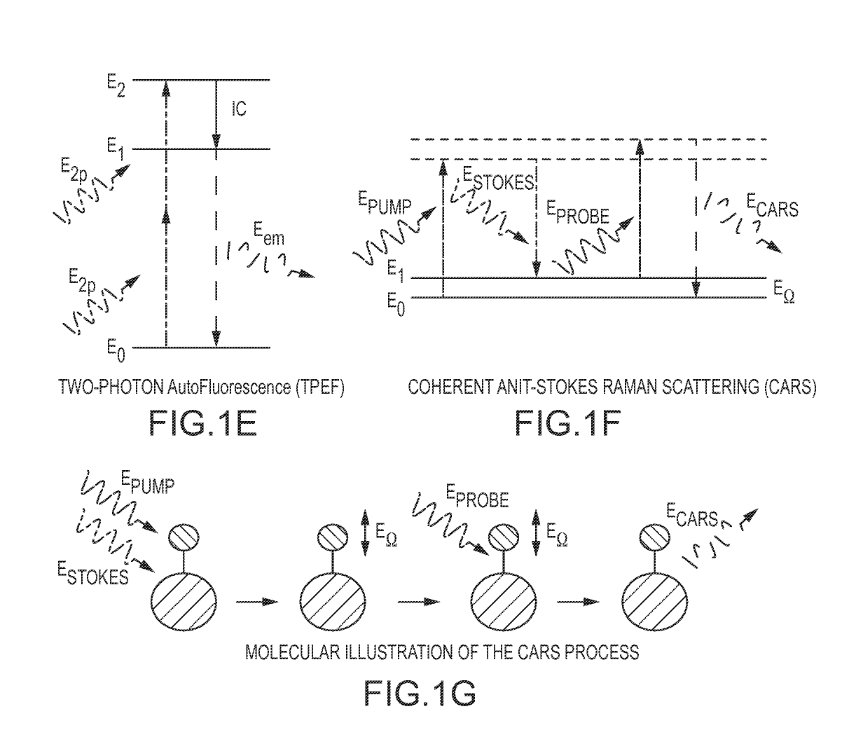

FIGS. 1A-1D show schematics of these different processes that result from nonlinear multi-photon interactions with a molecule. FIGS. 1A-1D are Jablonski diagrams showing the interaction of multiple infrared photons with the electronic and vibrational energy levels of a molecule. FIG. 1A shows TPEF is very similar to traditional one-photon fluorescence, except two photons of a lower energy hν₁ are simultaneously absorbed to excite a fluorophore. In two-photon excitation fluorescence (TPEF) the molecule absorbs two infrared photons that promote it to an excited electronic state. After relaxation to a lower vibrational level, the molecule emits a lower energy (red-shifted) photon having an energy hν_{2PEF}<2 hν₁. A fluorophore is any molecule that can absorb photons and emit the energy as a photon with a red-shifted wavelength.

FIG. 1B is a Jablonski diagram of second harmonic generation (SHG), another nonlinear process that occurs with two-photon excitation. In SHG, two infrared photons with energies hν₁ are instantaneously up-converted to a single photon of twice the energy, hν_{SHG}=2 hν₁. SHG only occurs when light interacts with non-centrosymmetric (asymmetric) macromolecular structures. Molecules such as collagen fibers can simultaneously “scatter” two lower-energy photons as a single photon of twice the energy.

FIG. 1C is a Jablonski diagram of third-harmonic generation (THG). THG is analogous to SHG; however, in this case, three photons of the fundamental are up-converted to a single photon of three times the energy hν_{THG}=3 hν₁. THG only requires about ten times the photon flux as SHG and therefore can be a useful tool for imaging. THG highlights different features of a sample than SHG because it is generated at the interface of media with differing third-order nonlinear susceptibilities, χ⁽³⁾.

FIG. 1D is a Jablonski diagram of coherent anti-Stokes Raman scattering (CARS). In CARS, two photons with energies hνₚ and hνₛ coherently excite the vibrational level with energy hΩ=hνₚ−hνₛ. An additional photon, hνₚ, interacts with the vibrationally excited molecule emitting a photon with energy given by the original incident photon energy plus the vibrational energy, hν_{CARS}=hνₚ+hΩ, leaving the molecule in its original ground state. (Note that photon energy is given by Planck's constant, h, multiplied by the frequency of the photon ν.)

FIGS. 1E-1F are energy diagrams of the Two-Photon Autofluorescence (TPAF) (FIG. 1E) and Coherent Anti-Stokes Raman Scattering (CARS) (FIG. 1F). The energy diagram for TPAF shown in FIG. 1E shows an autofluorecent molecule simultaneously absorbing two optical infrared photons (E₂ₚ). After internal-crossing (IC), in which some energy is lost, the fluorescent molecule will emit a fluorescence photon (Eₛₘₛ). In contrast, the energy diagram for CARS shown in FIG. 1F shows two optical photons with the photon energy difference (Eₚᵤₘₚ−Eₛₜₒₖₑₛ) equaling to the vibrational energy of a molecules (E_{Ω}) is used to excite the vibrational motion of the molecule. A third photon (E_{probe}) is subsequently used to excite the vibtational motion of the molecule, resulting in the emision of an energy-upshifted photon (E_{CARS}).

FIG. 1G shows a schematic diagram illustrating the CARS process. The pump and the Stokes photons simultaneously excite a lipid molecule, with the energy difference between the two photons equal to the vibrational energy of the molecule bond (E_{Ω}). Subsequent interaction of the probe photon coherently interacts with the vibrational motion of the molecule to generate a release of the CARS photon.

Endogenous fluorophores have varying two-photon cross sections as a function of wavelength and have been measured and reported. The center wavelength of a Ti:Sapphire laser can be tuned over a large spectral range from 700 to 1050 nm, making it an extremely useful source for two-photon autofluorescence excitation. In this manner, different compounds in tissue can be highlighted by tuning the excitation wavelength. For example, the two-photon cross-sections of many endogenous fluorophores peak below 700 nm and decrease at higher wavelengths while SHG emission remains strong at longer wavelengths from 900-1000 nm. By tuning to longer wavelengths, collagen structures in tissue can be distinguished from autofluorescence. In another example of the utility of excitation wavelength tuning, NAD(P)H was distinguished from FAD by excitation at 730 nm where both compounds are excited and at 900 nm where FAD is exclusively excited while NAD(P)H has a negligible two-photon cross section. Table 1 gives a list of endogenous fluorophores and tissue structures and example imaging techniques that provides contrast mechanisms.

TABLE 1
Example imaging contrast mechanisms for different biological molecules.

| Compound | Imaging technique (excitation/emission wavelengths) |
|---|---|
| NAD(P)H | TPAF (excitation 700-730 nm/emission peak 460 nm) |
| FAD | TPAF (excitation 700-900 nm/emission peak 525 nm) |
| Elastin | TPAF (excitation 700-740 nm/emission peak 400 nm) |
| Collagen | SHG (SHG excitation is tunable/emission at one half the |
| | excitation wavelength) |
| Lipids | THG/CARS (THG excitation is tunable/emission at one |
| | third the excitation wavelength) |

Fluorescence lifetime imaging microscopy (FLIM) is an additional imaging technique that is better able to distinguish between the different endogenous fluorophores in a biological sample. Due to the broad and overlapping emission spectra of many endogenous fluorophores, it can be difficult to quantitatively measure the concentrations of these different species contributing to the autofluorescence emission signal by spectral filtering alone. Fluorescence lifetime can also provide information on the surrounding environment of the fluorophore. FLIM is based on the fact that every fluorophore has a characteristic excited state lifetime, τ, or time for the molecule to decay from the excited electronic state to the ground state. This decay is characterized by a single or multiple exponential (in the case of an inhomogeneous environment) of the form:

$$P ( t ) = {P}_{0} {\sum }_{i = 1}^{n} {A}_{i} exp ( - t / {τ}_{i} ) ,$$

where P(t) is the population in the excited state as a function of time. Here, P₀ is the initial population in the excited state and Aᵢ is the normalized amplitude of the exponential component with lifetime τᵢ. Fluorescence lifetime signal from a biological sample containing multiple fluorophores can become further complicated. For multiple exponential lifetimes, the average lifetime value is sometimes reported, given by:

$$\overline{τ} = {\sum }_{i = 1}^{n} {A}_{i} {τ}_{i} .$$

This lifetime information can be measured either by time domain or frequency domain methods. In a time domain technique, a pulsed excitation source is used to excite the fluorophore of interest in the biological sample. The subsequent time profile of the fluorescence emission is measured using time gating techniques.

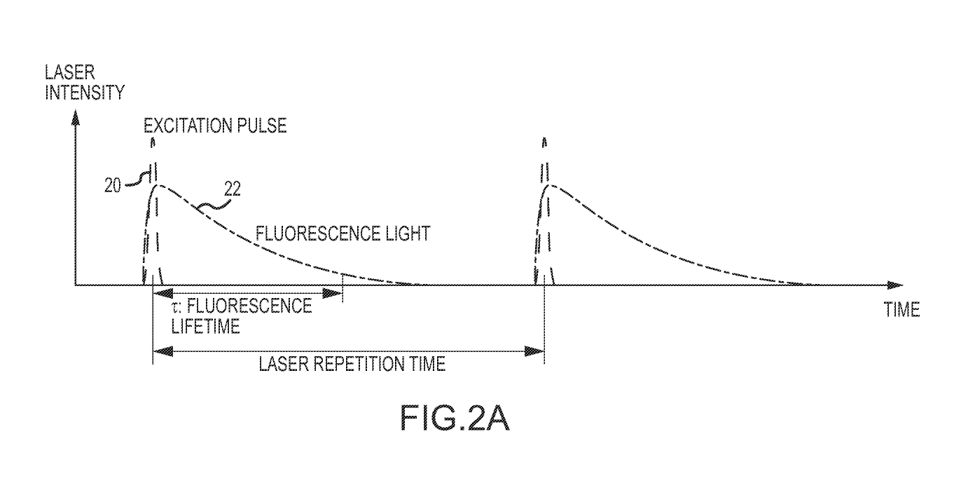

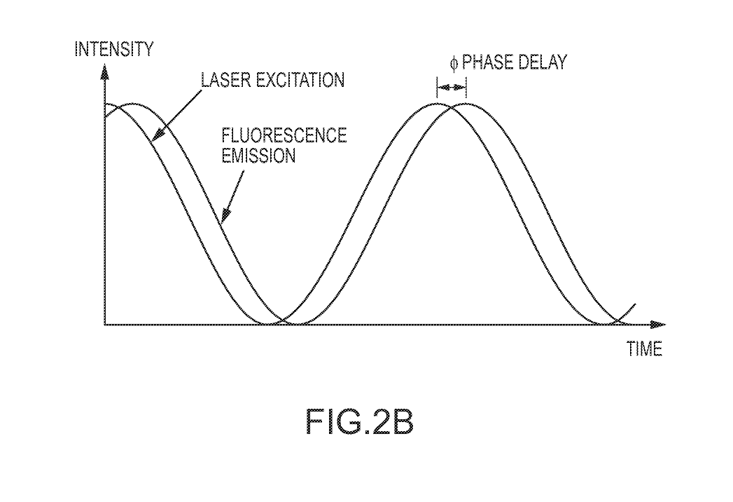

FIG. 2A illustrates a time-domain FLIM process. As shown in FIG. 2, a short pulsed excitation light 20 and a longer time duration fluorescence emission light 22 is shown as a function of time. In FLIM, the time scale of the fluorescence emission, τ, is measured.

FLIM has found particular use in imaging NAD(P)H. Bound and un-bound NADH have different characteristic lifetimes (free NADH ˜0.3 ns, protein bound NADH ˜2 ns) and therefore can be used to measure the ratios of these populations giving an indication of metabolic activity.

FIG. 2B is an illustration of frequency domain fluorescence lifetime measurement. The excitation light is modulated in amplitude at a frequency ω while the fluorescence light is emitted with the same modulation frequency but with a phase shift in time, φ. For a single exponential lifetime, the value of the fluorescence lifetime is related by tan φ=ωτ.

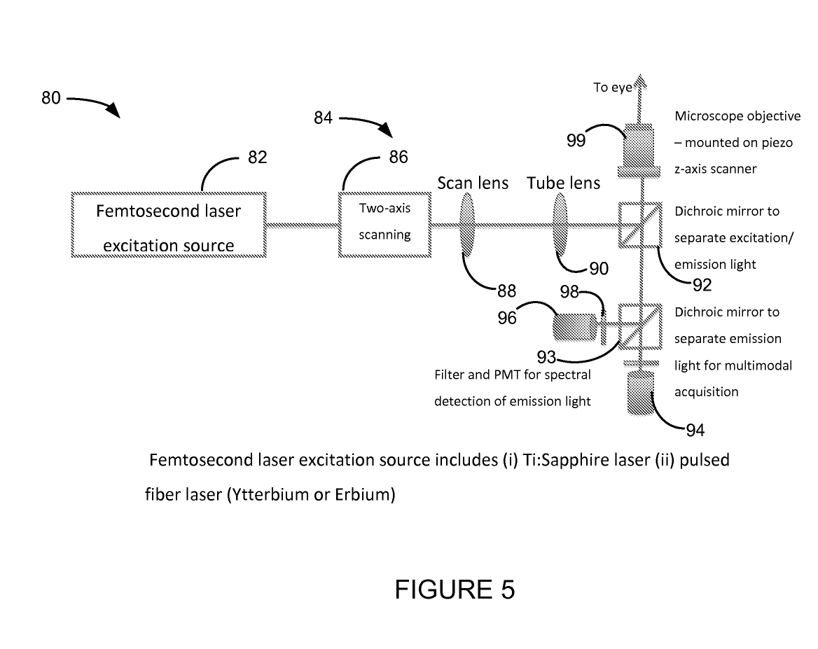

FIG. 2B shows frequency domain FLIM process. In frequency domain FLIM, an amplitude modulated excitation source is employed. The lifetime of the fluorophore causes the emitted fluorescence signal to be modulated at the same frequency but with a phase shift relative to the excitation light (see FIG. 5). Measurement of this phase offset using phase-sensitive detection (such as a lock-in amplifier) will then give the value of the lifetime, τ, by the relation, tan φ=ωτ, where φ is the phase offset, ω is the modulation frequency. If the lifetime is multi-exponential it is necessary to measure the phase offset at several modulation frequencies in order to obtain the different lifetime components. Some advantages of the frequency domain technique include faster acquisition compared to the time domain technique and insensitivity to high photon count rates, which is a problem with time domain techniques as high count rates can skew the time histogram to shorter times. Frequency-domain FLIM has been recently demonstrated using an inexpensive field programmable gate array and photon counting detection giving very rapid and highly sensitive measurements.

Optical Instrumentation

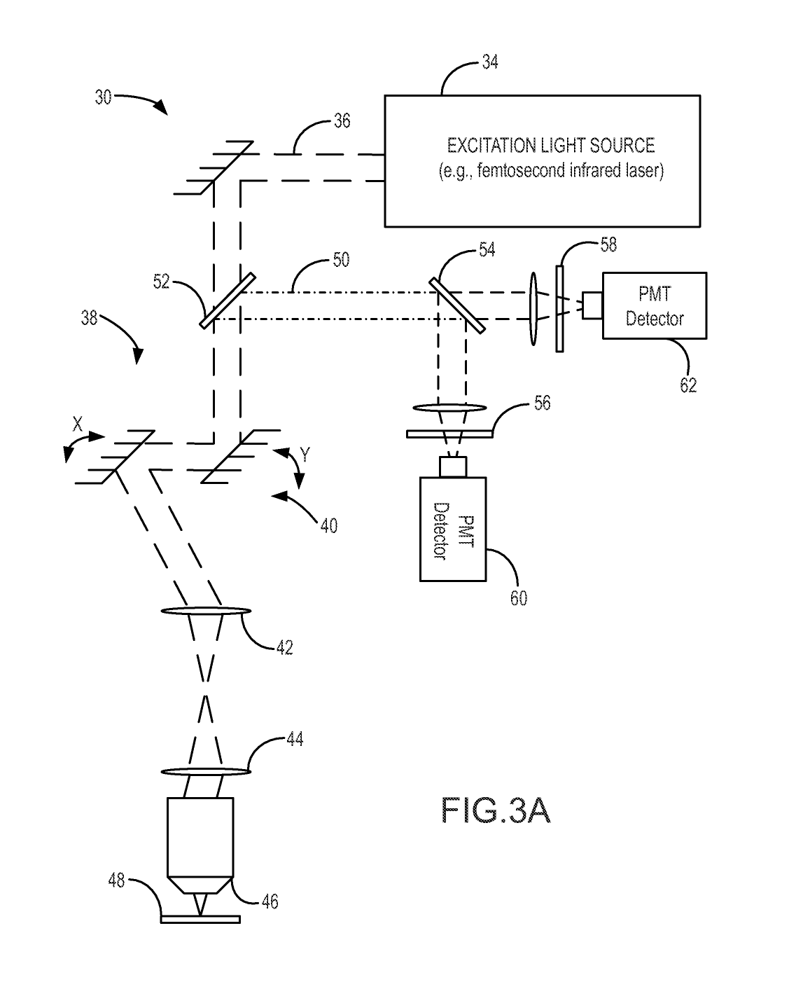

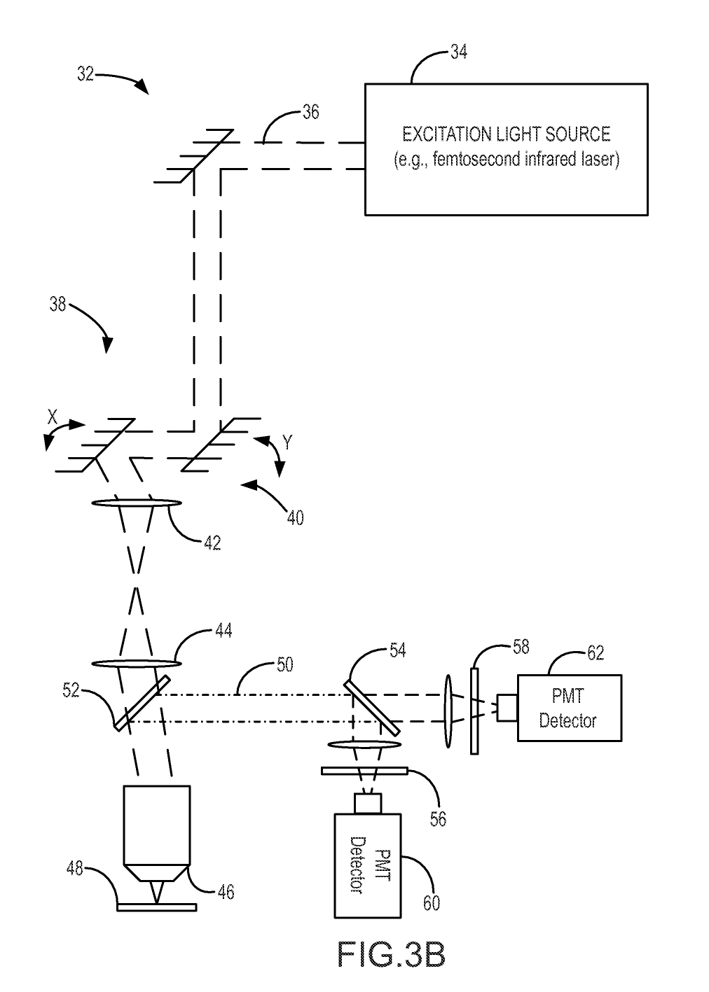

Different multi-photon microscopy imaging modalities (e.g., both TPEF and SHG) can be simultaneously measured using the same optical setup where signals of the respective modalities occur at distinct wavelengths. Spectral filtering can be used to separate the distinct wavelengths for the different imaging modalities. Both TPEF and SHG, for example, can be simultaneously measured using the same optical setup because the SHG signal occurs at a distinct wavelength (exactly half the excitation wavelength) and can be separated from autofluorescence using spectral filtering. FIGS. 3A and 3B show example schematics of a descanned imaging system 30 (FIG. 3A) and a non-descanned imaging system 32 (FIG. 3B) for performing multi-photon (MP) imaging. The apparatuses 30, 32 shown in FIGS. 3A and 3B each comprise an excitation light source 34 (e.g., a laser light source). In some embodiments, for example, the excitation light source comprises a pulsed femtosecond infrared laser source, such as but not limited to a Ti:Sapphire mode-locked oscillator. In one embodiment, for example, the excitation source comprises a 100 fsec, 80 MHz, 700-1050 nm Ti:Sapphire laser. In some embodiments, a femtosecond laser excitation source may comprise a femtosecond fiber laser. The femtosecond fiber laser, for example, may comprise a single mode femtosecond fiber laser, a photonic crystal fiber laser, a step index core laser, or a grading index femtosecond fiber laser.

Excitation light 36 is directed to and focused onto a sample 48 via an optical system 38. In the particular implementations shown in FIGS. 3A and 3B, for example, the excitation light 36 first passes through a two axis galvo-scanning minor stage 40 of the optical system 38 and is imaged, using a scan lens 42 and a tube lens 44, on to the back of a microscope objective 46. The microscope objective 46 focuses the light to a focal volume (e.g., around 200 nm axial and 1.0 microns lateral) depending upon the numerical aperture of the objective.

In the system shown in FIGS. 3A and 3B, the generated two-photon signal 50 is collected back through the same objective 46 and separated from the excitation light using a first dichroic minor DM1 52, a second dichroic mirror DM2 54, and filters 56, 58. The two-photon signal is then imaged onto the front of a photomultiplier tube (PMT) 60, 62. In the descanned detection system 30 (FIG. 3A), the multi-photon emission is relayed back through the galvo minor stage 40 so that the scanning motion is cancelled out and the emitted light is stationary at the detector 60, 62. In the non-descanned detection (FIG. 3B), the emission light is separated using a dichroic minor before passing through the scanning mirrors greatly reducing the loss in signal associated with reflections off of the minors and the lenses in the optical path. Because the two-photon emission is not passed back through the scanning minors in this embodiment, the emission light on the PMT moves during scanning. However, the PMT is typically insensitive to this motion because the large detection area. Non-descanned detection is available for multi-photon imaging because unlike in single photon confocal imaging, a pinhole is not required to eliminate out of focus light from the image.

Due to the limitations in the penetration depth, MPM has so far only been applied in the clinic for screening of the skin. The optical system shown in FIGS. 3A and 3B, however, can include microendoscopy optics for intrabody tissue imaging. The systems, for example, may be configured for probing of neural activity, blood flow measurements, imaging of goblet cells in gastric epithelium, detecting extracellular matrix proteins such as collagen and elastin in the human dermis.

In one embodiment, for example, the optical system may include compound gradient refractive index (GRIN) lenses as focusing optics, double-clad photonic crystal fibers for superior detection efficiency and mechanical flexibility, and/or microelectromechanical systems (MEMS) scanning mirrors. GRIN lenses, for example, have a typical size of 0.2-1 mm in diameter, 1-10 cm in length, and a numerical aperture of less than 0.6. Due to low numerical aperture and optical aberration, the optical Rayleigh resolution may be limited (e.g., to ˜1 μm in lateral and ˜10 μm in axial direction). In other embodiments, the optical system may comprise aberration-corrected, high NA plano-convex lenses (NA<0.85) acting like micro-objectives to provide on-axis resolution comparable to water-immersion objectives. The optical system may further take advantage of other microendoscopy technology to achieve multiphoton microscopy in intrabody clinical imaging.

Another clinical application of MPM is in histology where there is no requirement for deep tissue penetration as the tissue can easily be sectioned in 10-100 μm thick slices. MPM can have advantages over traditional histological staining techniques by providing more detailed information and highlighting features without perturbing the sample through processing. Preparation of samples for both standard histological staining and electron microscopy require chemical fixation and dehydration with alcohols. These treatments can cause artifacts and distortions within the tissue due to infusion of fixatives and shrinkage of tissue due to alcohol treatment. In additional, changes to fine tissue morphology can occur with heat-infusion of paraffin (for histology) or with polymerization of resin (EM).

Multi-Photon Eye Imaging

In one particular embodiment, multi-photon imaging is used to image an eye. Multi-photon imaging, for example, may be used to image sections of an eye or an intact eye. The mult-photon imaging, for example, may image an eye for disease identification, diagnostics, drug delivery monitoring, or the like. Although examples are provided for imaging eye tissue, the same techniques can also be used to image other types of tissue as well. For example, tissues such as skin, oral, and nasal cavities may be imaged using multi-photon imaging.

Glaucoma is one example of a disease that may be identified and/or tracked using multi-photon imaging technology. Glaucoma is the second leading cause of blindness in the United States affecting approximately 3 million adults. Worldwide, the numbers are estimated to increase to 60 million by 2020. Glaucoma most often occurs in people over age 40, although a congenital or infantile form of glaucoma also exists. While glaucoma is a neurodegenerative disease (a disease involving loss of nerve cells in the eye), the primary problem is loss of proper fluid flow out of the eye's drainage system. This leads to an increase in eye pressure, known as an increase in intraocular pressure (TOP). It is important to realize that not all patients with glaucoma have an obvious increase in IOP, and that some patients with high IOP don't necessarily have glaucoma. IOP is just a measurement that helps identify people at risk for developing the disease. Current ways to diagnose glaucoma include 1) checking peripheral vision with a “Visual Field Machine”, 2) examining the thickness of the retina and nerve in the back of the eye (known as the optic nerve) for loss of tissue that results from loss of nerve cells, 3) checking IOP with an eye pressure machine known as a tonometer. None of these tests can measure the workings of the actual drainage system of the eye. This is why we believe there is a great need for new devices that can diagnose glaucoma by directly measuring the tissue in the drainage system for any signs of problems. The tissue in the drainage system is known as trabecular meshwork (TM).

In one embodiment, multi-photon microscopy (MPM) imaging can be used to image one or more regions of the eye, such as regions of the eye implicated in a variety of disease pathologies. Current clinical techniques for imaging include optical coherence tomography (OCT) and confocal reflectance microscopy as well as fluorescence imaging. In comparison with MPM imaging, OCT imaging has poorer spatial resolution of 2-10 μm and therefore cannot be used to reveal sub-cellular level structure. While confocal reflectance microscopy does allow sub-cellular level resolution, its contrast mechanism is due to changes in index of refraction and therefore it does not have the functional information inherent in MPM imaging. Fluorescence imaging uses exogenous dyes to stain the eye in a non-specific manner typically for looking at the vasculature in the retina. None of these approaches are capable of providing functional data for imaged tissues and are thus limited in their ability to direct or influence clinical decision making on a consistent basis. Although these approaches have limitations, multi-photon approaches may be used in combination with these other approaches.

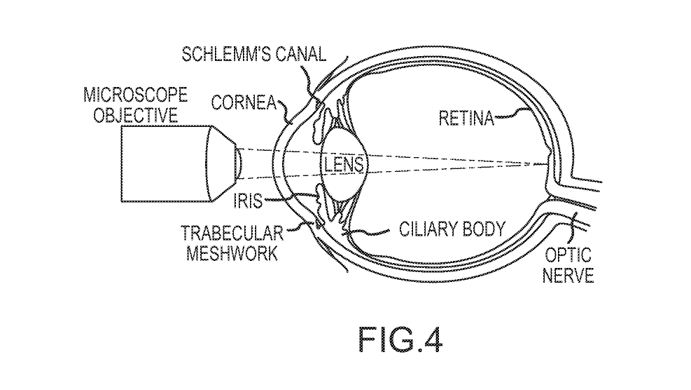

FIG. 4 shows a diagram of an eye highlighting example regions for MPM imaging. MPM imaging of the cornea, for example, is of interest for diagnosis of diseases such as corneal dystrophies and endothelial dysfunction and has been reported by several groups. Multi-photon imaging approaches have also been reported on various regions of the eye. TPAF, SHG, and autofluorescence lifetime imaging of different ocular surface pathologies, for example, have been performed using a commercial instrument for clinical multi-photon imaging (DermaInspect, JenLab GmbH, Neuengonna, Germany). By performing multiple wavelength excitation at 730 nm and 835 nm and resolving different lifetime components by FLIM, epithelial cells, goblet cells, erythrocytes, macrophages, collagen, elastin, vascular structures, and pigmented lesions have been identified and distinguished between. SHG, TPAF and THG of the cornea and SHG and TPAF of the trabecular meshwork have been demonstrated. In particular, an additional contrast mechanism has been demonstrated by selecting either linear or circularly polarized excitation for THG. Simultaneous reflectance confocal microscopy, TPAF, and SHG on corneal sections have also been demonstrated. 3D SHG imaging has also been used to characterize structural lamellar organization of the anterior cornea. Simultaneous SHG and TPAF imaging has been demonstrated to identify cellular components of the cornea, limbus, and conjunctiva, as well as imaging corneal and scleral collagen fibers. MP imaging of both cornea and retinal sections has also been demonstrated.

MPM imaging of the retina has also been demonstrated and may find utility in detection of retinal pigment epithelium (RPE) dysfunction and photoreceptor related dystrophies. To date, no imaging of the retina has been performed through the anterior chamber, although explants of human retina and RPE have been imaged by the tissue autofluorescence. There are additional difficulties in imaging the retina for clinical applications due to the optical constraints posed by the iris that effectively limit the numerical aperture. For example, for an iris opening of 8 mm diameter and typical distance from iris to the retina of 17 mm, the effective numerical aperture, which is indicative of the collection angle of the emitted optical signal, is given by the equation NA=n sin θ˜0.3, using the index of refraction of water (n=1.33). The numerical aperture also limits how tightly the excitation light can be focused. In addition, the aberrations in the lens of the eye can also decrease the obtainable resolution using multi-photon imaging. In order to alleviate this problem, wavefront correction using adaptive optics has been performed for retinal imaging.

To our knowledge, MPM imaging of a living retina/RPE has only been performed in a rodent eye by imaging through the exterior sclera. In this instance, Imanishi et al used MPM to view the retina/RPE autofluorescence as well as to localize stores of the visual pigment retinal. See Imanishi, Y., et al., Noninvasive two-photon imaging reveals retinyl ester storage structures in the eye. J Cell Biol, 2004. 164(3): p. 373-383. The retina itself has no apparent SHG signal, although the overlying retinal vasculature and underlying connective tissue can be imaged via the collagen content. The present inventors have demonstrated this in a lab on a Zeiss LSM510 multiphoton confocal microscope 80, illustrated in FIG. 5. An excitation light source, in the embodiment shown in FIG. 5, comprises a femtosecond laser excitation source 82. In one particular implementation, the femtosecond laser excitation source includes a Ti:Sapphire laser and a pulsed fiber laser (e.g., Ytterbium or Erbium). An excitation signal is passed through an optical system 84 and is focused onto a sample. In the particular embodiment shown in FIG. 5, for example, the optical system 84 comprises a two-axis scanning system 86, a scan lens 88, and a tube lens 90 for scanning the excitation signal across the sample. A pair of diachronic minors 92, 93 of the optical system 84 pass the excitation signal to the sample via a microscope objective 94. An excitation signal is received from the sample via the objective 94 and is directed to the dichroic minor 93 that separates components of the emission signal for multi-modal acquisition. In the embodiment shown in FIG. 5, for example, a first component of the emission signal is separated from via the dichroic minor 93 and directed to a detector 96 (e.g., a photomultiplier tube) via a filter 98 for a first mode of acquisition. A second component of the emission signal is separated from the excitation signal via the dichroic minor 92 and is passed to a microscope objective 99 mounted on a piezo z-axis scanner for a second mode of acquisition.

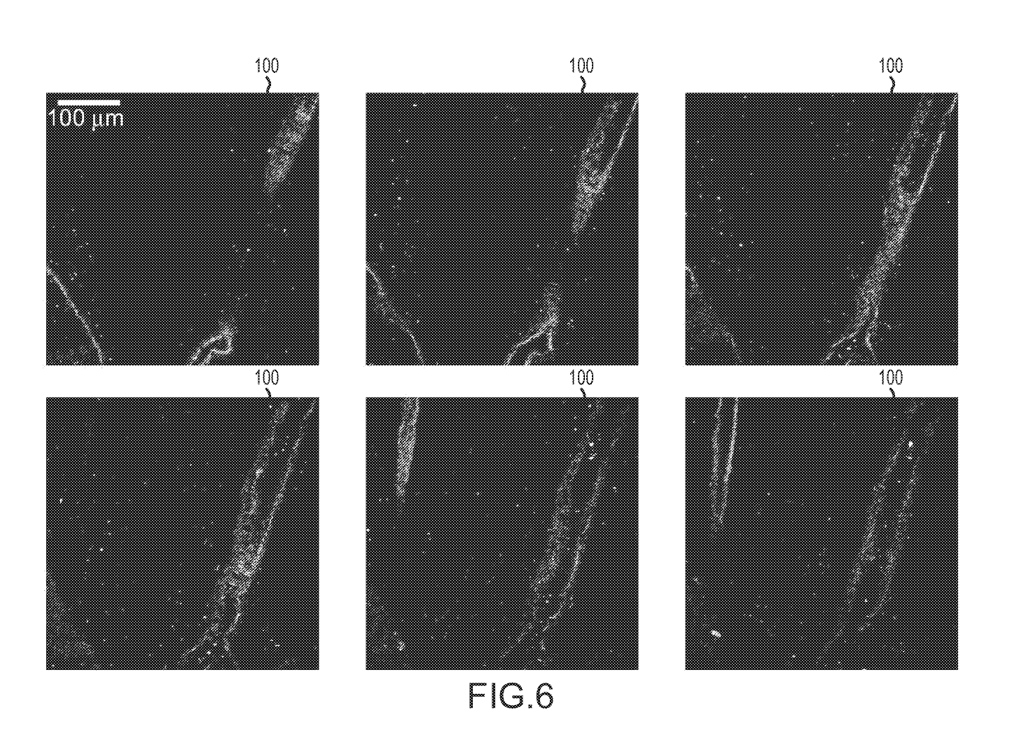

FIG. 6 shows a vascular bed of a human retina imaged by second harmonic generation (SHG). Serial z-sections, spaced 12 μm apart, of a human retina are shown beginning with the upper left panel through lower right panel. The images shown were collected using the 800 nm near infrared laser excitation with a collection window of 390-410 nm. The collagen structure of a large blood vessel 100 is clearly visible through the series, which represents a height of 60 μm. Starting in the upper left panel and traveling to the lower right panel of FIG. 6, one can see the top of a blood vessel followed by the inside of the vessel as the objective moves through the vascular bed.

Trabecular Meshwork

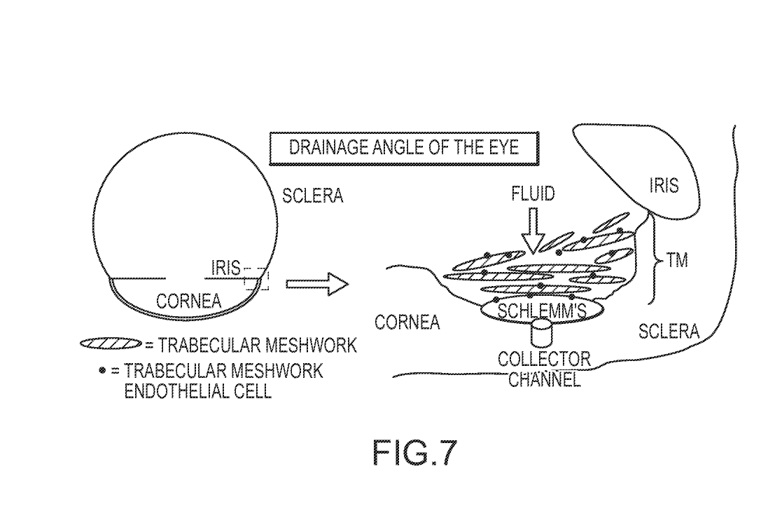

In the conventional outflow system of the eye, aqueous humor exits the anterior chamber through the trabecular meshwork (TM) before passing through Schlemm's canal. This region is characterized by overlapping collagen bundles that create a porous tissue populated by TM endothelial cells. These cells have been implicated in maintaining the health of the TM, the number of live TM cells within the meshwork was found to be statistically lower in patients with primary open-angle glaucoma (Alvarado Ophthalmology 1984; 91(6):564-579). The trabecular meshwork (TM) lies just outside of the circumference of the cornea, below the outer edge of the scleral region of the eye as shown in FIG. 7. The hallmark indicator of glaucoma, elevated intraocular pressure (TOP), is believed to result from dysfunction of exit of vitreous humor from the eye through this region. The TM is composed of multiple layers of extracellular matrix populated by trabecular meshwork endothelial cells (TM cells). Normally, fluid filters through this meshwork, into Schlemm's canal, and then drains from collector channels located within the sclera into the episcleral venous system.

Currently, neither optical coherence tomography (OCT) nor an ultrasound biomicroscope can image the TM/Schlemm's canal region with fine enough resolution to either diagnose or follow the progression of glaucoma. OCT uses low-coherence light interference to generate cross-sectional images of the eye with a 10 micron axial resolution and 20 micron transverse resolution. An ultrasound biomicroscope has similar resolution (˜25 microns) with better ability to detect small density differences. Neither method, however, has the resolution to image the conventional outflow pathway (Schlemm's canal, collector channels), or the ability to distinguish TM cells from the surrounding extracellular matrix.

Multiple embodiments have demonstrated imaging the TM using MPM imaging. In one embodiment, for example, using Two-Photon AutoFluorescence (TPAF) and Second Harmonic Generation (SHG) nonlinear optical microscopy, the collagen fibrils in the TM can be readily imaged without the needs of exogenous fluorophores. See, e.g., Ammar D A, Lei T C, Gibson E A, Kahook M Y. Two-photon imaging of the trabecular meshwork. Mol Vis. 2010; 16:935-44. PMCID: 2890557; and Gibson E A, Masihzadeh O, Lei T C, Ammar D A, Kahook M Y. Multiphoton microscopy for ophthalmic imaging. J. Ophthalmol. 2011; 2011:870879. PMCID: 3022205, each of which is incorporated herein in its entirety.

In another embodiment, Two-Photon AutoFluorescence (TPAF) can be used to visualize the endogenous NADP(H) of living human trabecular meshwork cells (TM cells), and map the response of these cells to oxidative stress. See, e.g., Masihzadeh O, Ammar D A, Lei T C, Gibson E A, Kahook M Y. Real-time measurements of nicotinamide adenine dinucleotide in live human trabecular meshwork cells: Effects of acute oxidative stress. Exp Eye Res. 2011, which is incorporated herein in its entirety. This process may be adapted to imaging TM cells within the eye.

In another embodiment, using CARS microscopy further allowed imaging TM cells that reside in the collagen mesh structure in the TM. In this embodiment, TM cells were readily imaged without exogenous labeling at the corneal rim of a human cadaver eye.

SHG and TPAF

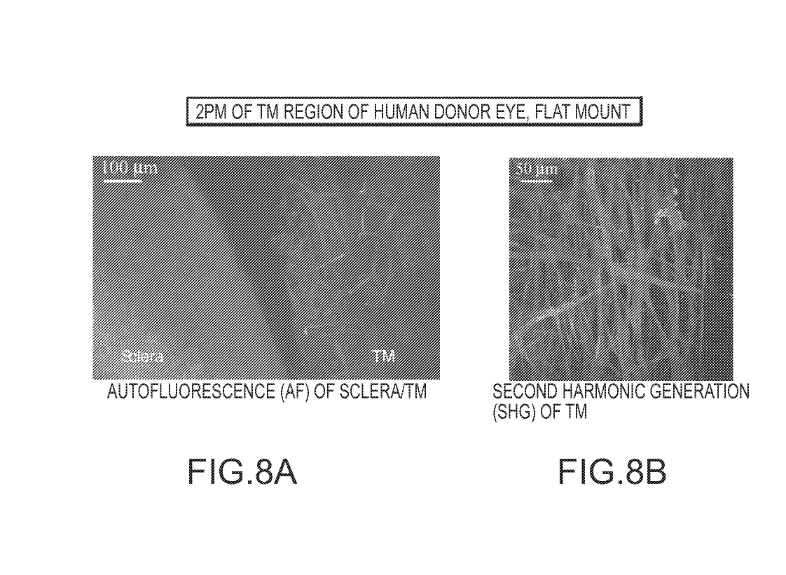

In one example embodiment of an imaging system, a commercial Zeiss LSM510 multiphoton confocal microscope with an LCI “Plan-NeoFluar” 25× objective (Zeiss) with a 0.21 mm working distance was used. While it was unable to perform trans-sclera imaging, TPM was successfully performed on a human eye with the TM flat-mounted toward the objective lens. TPAF from the extracellular matrix was imaged in the TM and scleral strip [FIG. 8A] as well as SHG from collagen within the TM shown in FIG. 8B, which appears as a meshwork of ˜10 micron thick fibers.

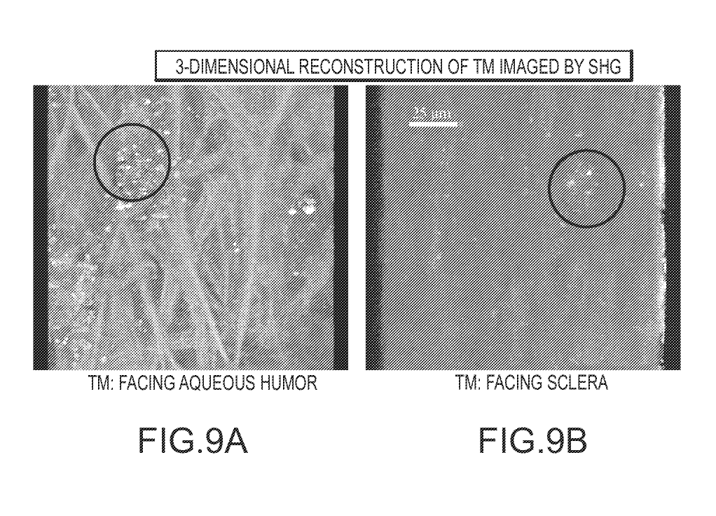

Further analysis on a similar region shown in FIG. 8B was also performed. Multiple z-sections of flat-mounted TM were imaged for SHG, and then computer modeled to show the 3-dimensional structures. A 60 micron deep region of TM was visually sectioned at 0.75 micron intervals to generate the images in FIGS. 9A-9B. This depth encompasses the TM/Schlemm's canal region and potentially the surface of the sclera. The aqueous humor face of TM (FIG. 9A) shows numerous collagen fibers by SHG. Rotation of the three-dimensional structure by 180 degrees (FIG. 9B-sclera face) shows an open collector channel like structure (circled) that penetrates the entire 60 microns of tissue. A non-fibrous structure present at the aqueous surface can be viewed through it.

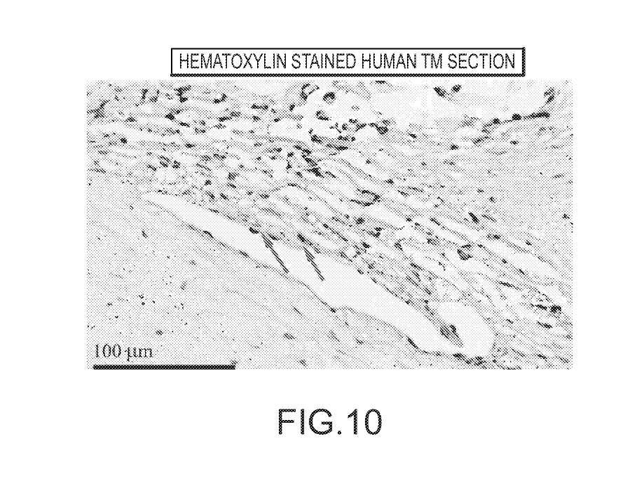

Comparing the fibrous structures seen by TPM with the structures seen in standard histological section of the TM region of the eye FIG. 10, the meshwork of collagen fibers of the TM appears as a honey-comb like structure above the open region of the Schlemm's canal. Hematoxylin-stained nuclei appear as purple structures (left arrow), while melanin present in the TM appear as brown granules of various sizes (right arrow).

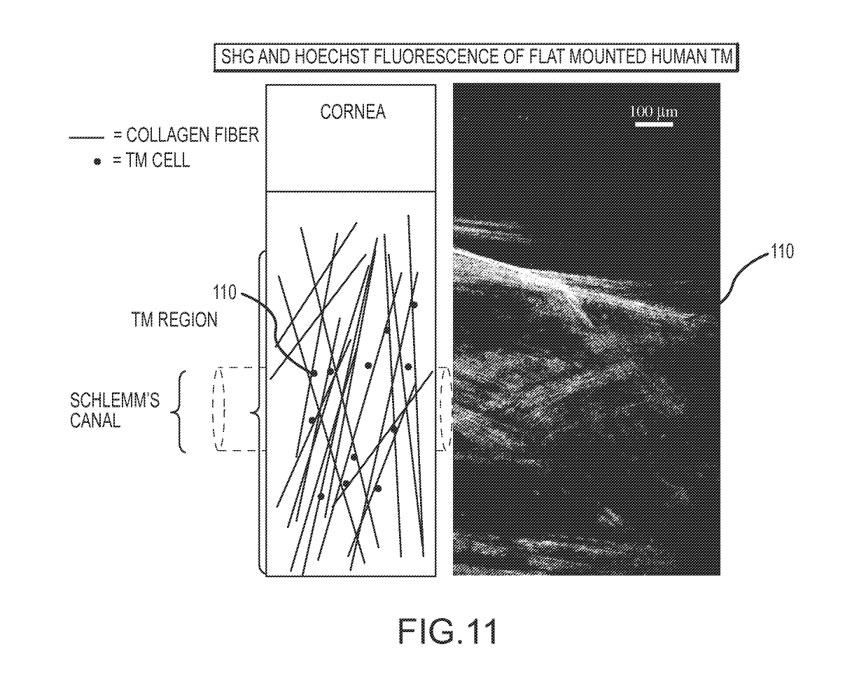

Towards the goal of imaging TM cells within unfixed tissue, another experiment was performed in which a fluorescent nuclear stain (Hoechst 33342) was injected into the anterior chamber of an intact donor eye. The eye was then opened, and a section of TM was flat-mounted facing the microscope objective. The TM cell nuclei 110 was imaged using standard two-photon excitation (TPEF) of the fluorescent dye. The collagen fibers within the TM were imaged by SHG 112 (lighter color fibers) as shown in FIG. 11. This figure represents a single z-plane within the TM, created by stitching together several overlapping images. At this magnification, the long collagen fibers are just visible, organized into multiple bundles of parallel strands. There are regions of both tightly overlapping bundles, but also empty regions that may represent fluid channels, possibly collector channels. These open regions are not to be confused with the Schlemm's canal, which would be below this plane, oriented perpendicular.

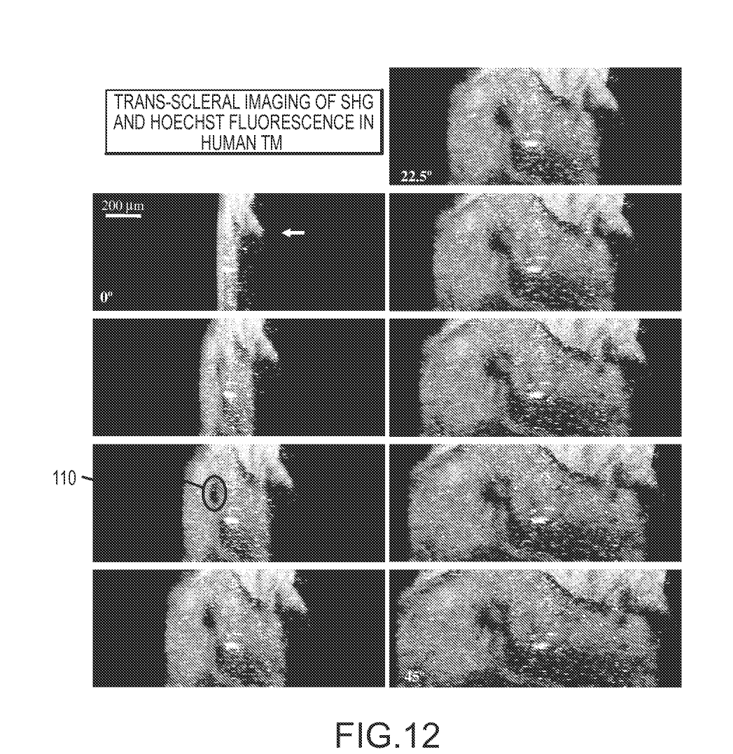

Another imaging example used a long-working distance 20× objective lens (Zeiss LD “Plan-NeoFluar”), with NA of 0.4 and a working distance 7.9 mm. This allowed imaging of a human eye through the sclera (sclera mounted facing the objective lens). Once again, the TM cell nuclei (blue) are imaged by standard TPM and the collagen fibers of the TM (white) imaged by SHG. FIG. 12 shows a three-dimensional reconstruction as a series of images at various angles of rotation (0° to 45°), starting from viewing the eye at cross section. The aqueous region is located on the right, and what is likely to be the scleral spur is visible at the arrow (←). A collector channel becomes visible as the tissue rotates (circle 114).

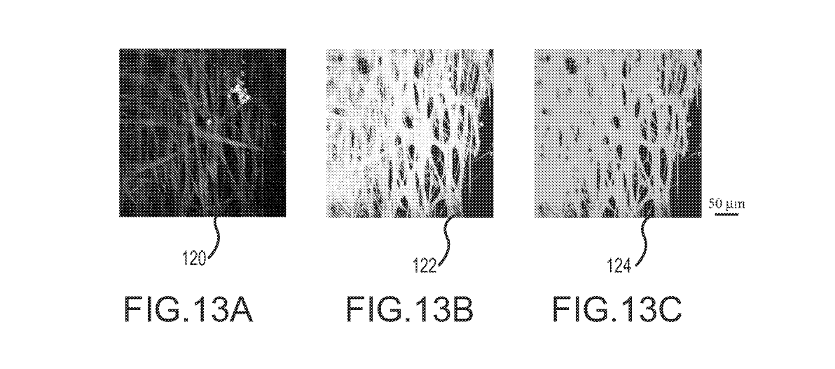

In another example embodiment, multi-photon imaging of the TM region of an eye was demonstrated using SHG and TPAF. See, e.g., Ammar, D., et al., Two-photon imaging of the trabecular meshwork. Molecular Vision, 2010. 16: p. 935-944, which is incorporated by reference herein in its entirety for all that it teaches and discloses. Imaging of the TM region of the eye is important because degeneration of the TM is implicated in glaucoma, therefore characterizing the cell and collagen structures in the TM may allow early diagnosis, disease monitoring, as well as fundamental studies of the disease mechanism. FIGS. 13A-13C show second harmonic generation (SHG) and two-photon autofluorescence (TPAF) of the TM region of a human eye from a 73 year old donor. A section of the eye was flat-mounted with the anterior chamber facing a microscope objective. The TM was visually sectioned by 0.5 μm intervals to a depth of 50 μm and then computer modeled into a single-plane projection (FIGS. 13A-13C). The images shown in FIGS. 13A-13C represent a projection of the multiple z-sections flattened into a single plane. SHG and TPAF emission windows were collected using the META spectral detector on a Zeiss LSM510 multiphoton confocal system. FIGS. 13A and 13B show the SHG 120 and TPAF 122 fluorescence, respectively. FIG. 13A shows the SHG emission (388 nm to 409 nm) collected from 800 nm excitation of TM. FIG. 13B shows a TPAF emission window (452 nm to 644 nm) collected simultaneously. FIG. 13C shows a merged image 124 of SHG 120 and AF 122 emission. Black scale bar=50 μm. This figure is reprinted from Ammar D A, Lei T C, Gibson E A, Kahook M Y. Two-photon imaging of the trabecular meshwork. Mol Vis 2010; 16:935-944, which is incorporated by reference herein in its entirety for all that it teaches and discloses.

Although the SHG signal is comparatively weaker than the TPAF, these two signals are qualitatively the same when overlapped in FIG. 13C. In one implementation, the two signals can be color-coded on the same image (e.g., blue=SHG, green=TPAF). Since collagen is the most common non-centrosymmetric macromolecule in the TM, the SHG signal is highly suggestive that the structures seen by TPAF are in fact collagen fibers. In these images of the TM, the majority of collagen fibers of the TM appear as smooth bundles of between 10 and 20 μm, although the occasional ˜1 μm collagen fiber is visible. These bundles have a fairly consistent diameter over short distances, but over longer distances (>250 μm) commonly split or join other bundles. The end result is a meshwork of collagen interwoven with varying-sized regions of non-fluorescent signal, which is assumed to be fluid spaces.

CARS and TPAF

In another implementation, the MPM imaging of the eye, such as the trabecular meshwork (TM) of the eye is performed via a combination of coherent anti-Stokes Raman scattering (CARS) and one or more other MPM imaging technique, such as two-photon autofluorescence (TPAF). In this implementation, flat-mounted trabecular meshwork samples from human cadaver eyes were imaged using CARS and TPAF non-linear optical techniques. In TPAF, two optical photons are simultaneously absorbed by autofluorescence molecules such as collagen and elastin. The CARS technique uses two laser frequencies to specifically excite carbon-hydrogen bonds, allowing the visualization of lipid-rich cell membranes. Multiple images were taken along an axis perpendicular to the surface of the TM for subsequent analysis.

Analysis of multiple TPAF images taken at various distances beneath the surface of the TM revealed the characteristic overlapping bundles of collagen of various sizes. Simultaneous CARS imaging revealed round structures of 10.3±1.2 microns by 6.9±1.1 microns in diameter populating the meshwork that appeared to be TM cells. Irregularly shaped objects of 4.2±0.6 microns by 3.2±0.4 microns appeared in both the TPAF and CARS channels, and are assumed to be melanin granules. In this example, CARS imaging allowed imaging of live TM cells in freshly isolated human TM samples.

In this implementation, a human globe was obtained from the San Diego Eye Bank (San Diego, Calif.). Approval was obtained from the Colorado Multiple Institutional Review Board for the use of human material and the tenets of the Declaration of Helsinki were followed. Informed consent was obtained from donor or relatives for use in research. Eyes were from a pseudophakic 86 year old donor with no history of glaucoma. The intact globe was cut circumferentially approximately 3 mm from the corneal limbus. This anterior region was cut into quadrants, and the overlying ciliary body and iris was cut away from the TM region using spring scissors. This quadrant of corneal rim tissue was placed in a glass-bottom 35 mm dish (MatTek Corporation; Ashland, Mass.) with the interior surface facing down. A small glass weight placed on top of the corneal rim to maintain contact of the tissue with the glass coverslip.

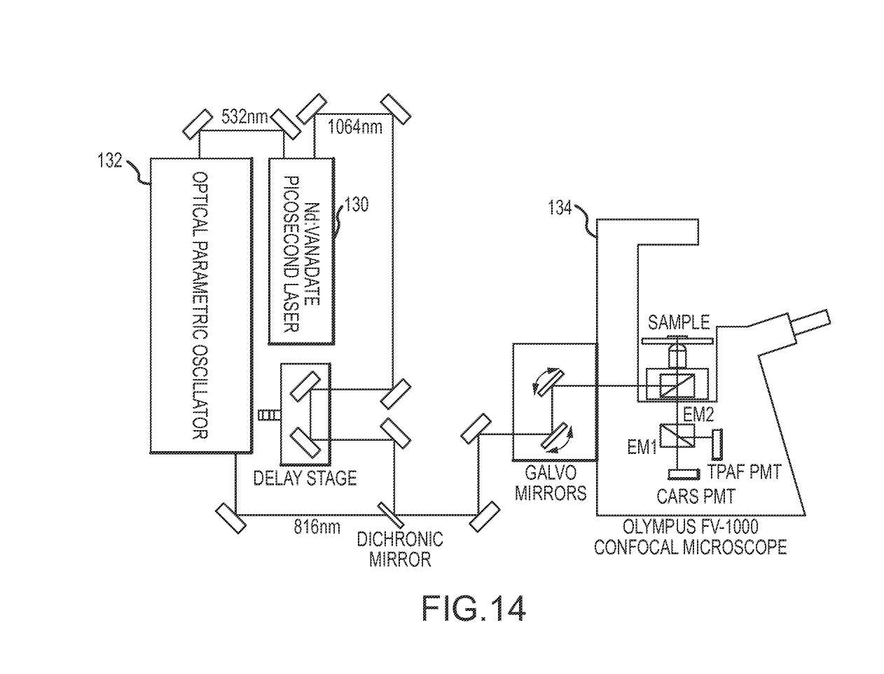

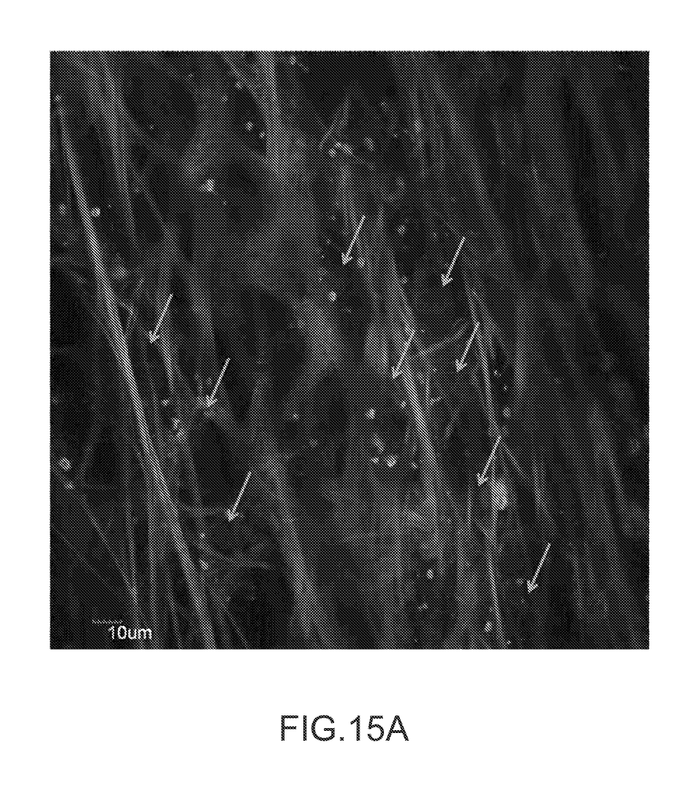

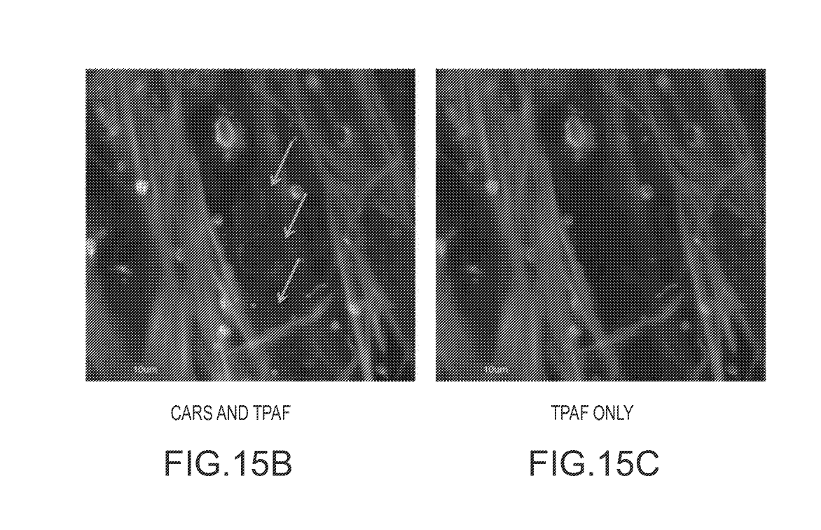

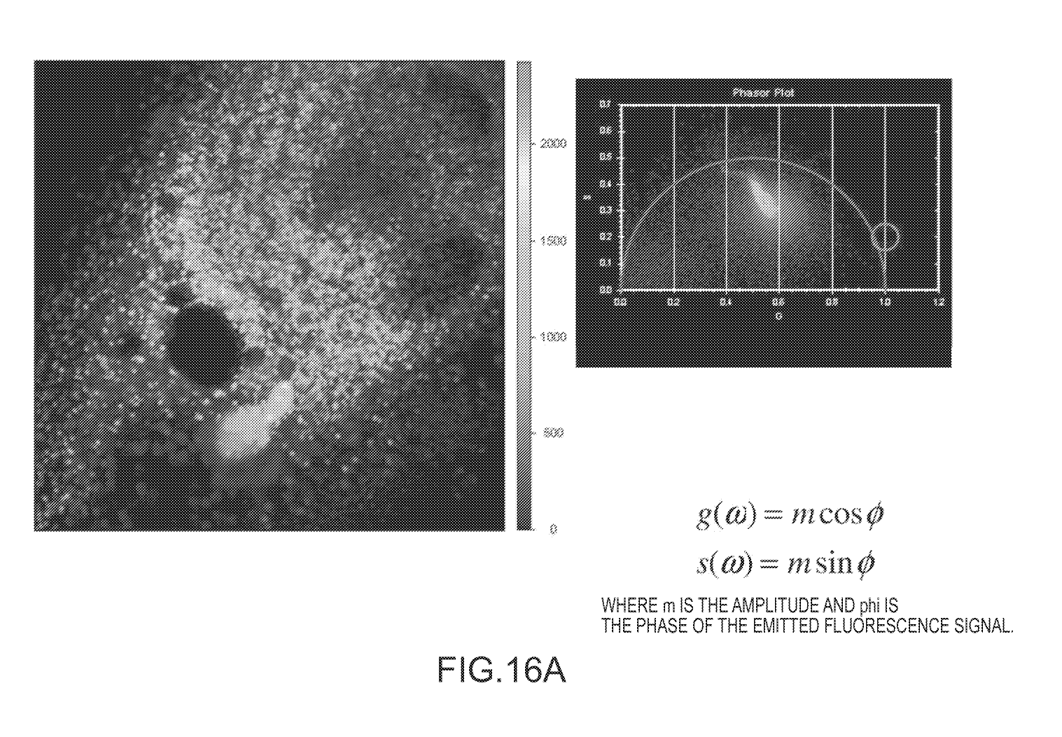

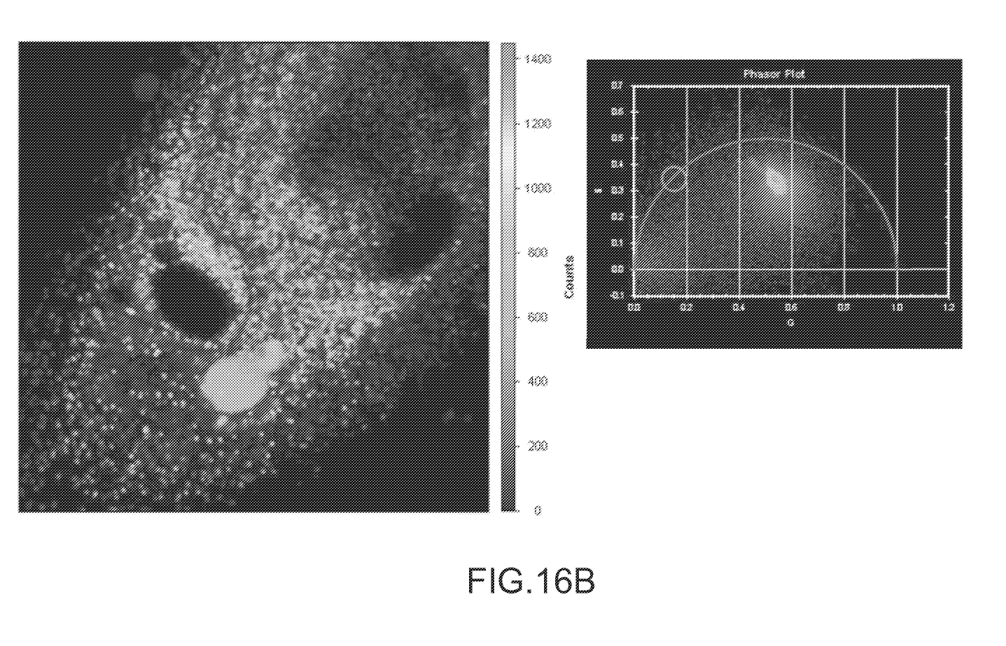

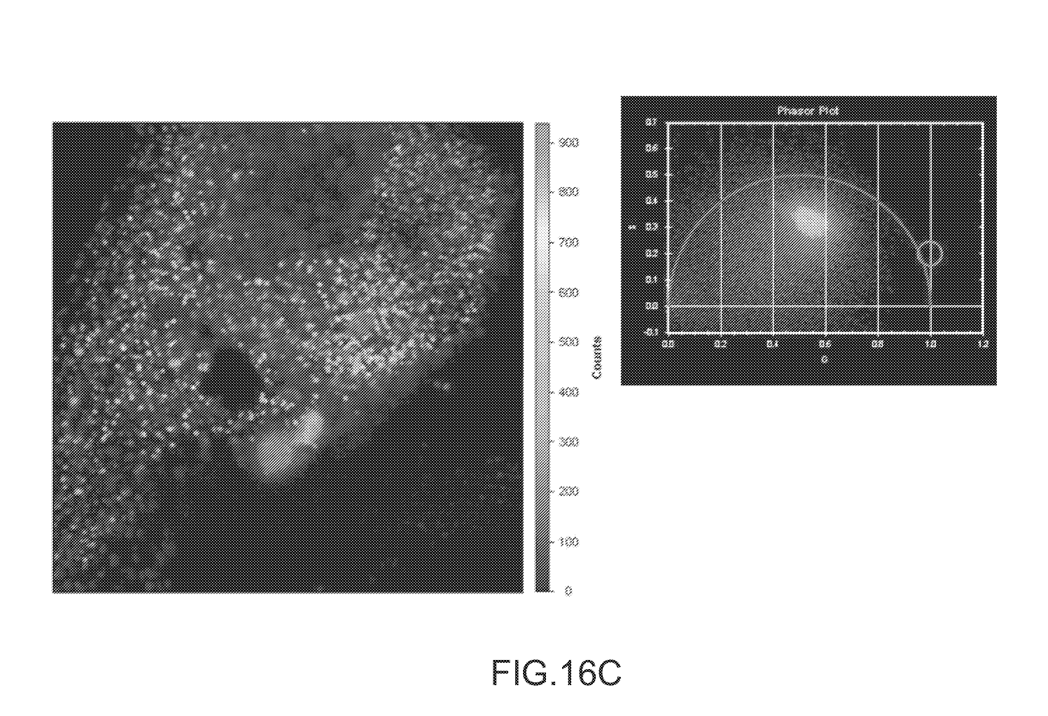

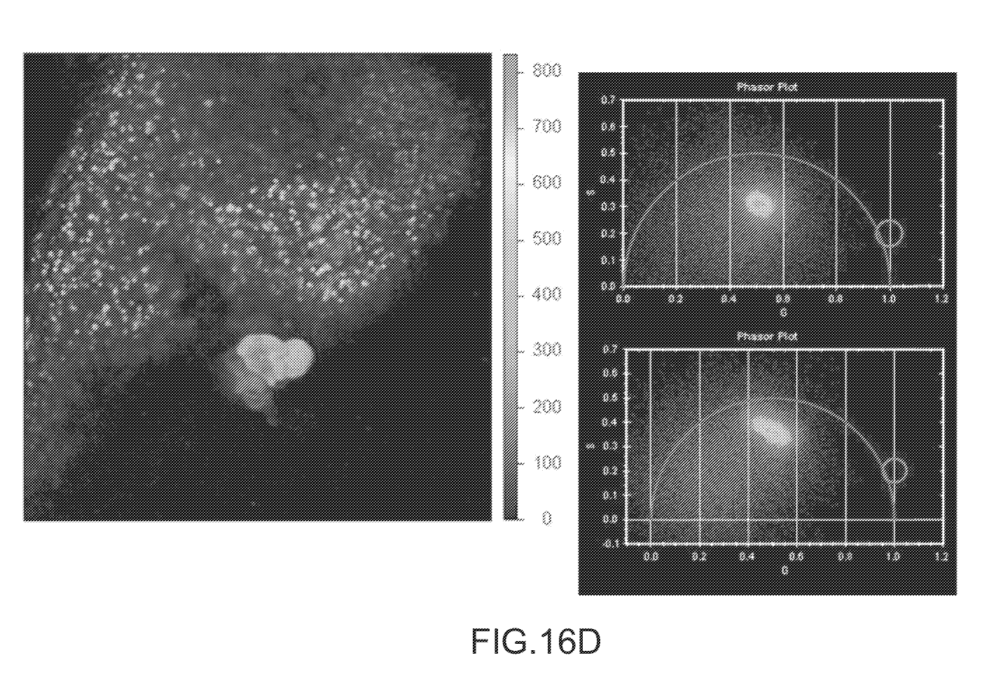

The CARS/TPAF images of the cells in the TM of the corneal rim of a cadaver eye was acquired with a custom-built multi-photon microscopy platform shown in FIG. 14 optimized for CARS and TPAF imaging as shown in FIG. 15A-15C. The system comprises a diode-pumped Nd:Vanadate (Nd:YVO₄) picoseconds (ps) laser 130 (picoTRAIN, HighQ Laser, Austria) capable of generating 10 Watt of 1064 nm of ˜7.5 ps optical pulses at a repetition rate of 80 MHz. Inside the laser, 9 Watt of the generated 1064 nm laser beam is redirected to a frequency doubling crystal to produce 4 Watt of 532 nm with ˜6 ps optical pulsewidth. The 4 Watt 532 nm laser beam is subsequently sent into an optical parametric oscillator 132 (Levante Emerald, APE, Germany) to convert the 532 nm laser beam into a 1 Watt, ˜6 ps, 816 nm laser beam through the nonlinear optical process of difference frequency generation. The remaining 1W 1064 nm beam (Stokes) from the Nd:Vanadate laser is then optically recombined with the 816 nm optical beam (Pump and Probe) and the combined laser beam is sent into an Olympus FV-1000 confocal microscope platform 134 for CARS and TPAF imaging. The Olympus FV-1000 microscope 134 is an inverted microscope and is equipped with four external detectors—two detectors in the epi-direction (e.g., non-descanned detectors) and the other two detectors in the forward directions. EM1 is an emission filter to allow autofluorescence signal from ˜420 to 520 nm to be detected by the TPAF PMT and EM2 is an emission filter to detect the CARS signal at 662 nm by the CARS PMT detector. In this experiment, both the TPAF and CARS signals are measured in the epi-direction by collecting back-scattered photons through the objective. A dichroic minor is used to separate the TPAF signal from the CARS signal and detected by the two epi-detectors respectively. Using an emission filter (hq470/100m-wp, Chroma Technology) in front of the first epi-detector, autofluorescence signal between 420 to 520 nm is detected. The CARS signal is measured with the second epi-detector with an emission filter centered at 660 nm. (hq660/40m-2p, Chroma Technology) The objective used in this experiment is a 60×1.2NA water objective (UPLSAPO 60×IR W, Olympus) optimized for CARS and TPAF imaging. The pixel dwell time is 10 μs and the image pixel resolution is 1600×1600 for all the acquired images. A Kalman average filter of 5 times is used during image acquisitions to improve the signal-to-noise ratio of the acquired images.

CARS/TPAF images are taken along the TM region in the cadaver coronal rim sample. FIG. 15A shows an example label-free image of a TM region of a human cadaver eye using two-photon autofluorescence and CARS, displaying the TPAF imaging channel in green and the CARS imaging channel in red. Due to autofluorescence of the collagen molecules, the collagen extracellular matrix shows clearly in the TPAF channel. In these images of the TM, the collagen fibers appear as smooth fiber bundles of various diameters, ranging from 1 and 10 μm. The fibers are straight with a consistent diameter, although the occasional bifurcation is visible. Qualitatively, the fiber structures are similar to those seen previously using TPM. See Ammar D A, Lei T C, Gibson E A, Kahook M Y. Two-photon imaging of the trabecular meshwork. Mol Vis. 2010; 16:935-44. PMCID: 2890557. In addition, the cell membrane of the TM cells is picked up in the CARS channel. These cells are shown residing in the interstitial region between the collagen fiber structure (FIG. 15A, arrows). The size of the TM cells shown in the image is approximately about 10 μm, which is the expected size of TM cells.

In FIGS. 15B-15C, the scanning magnification of the image has been increased 3 times using the 60× objective to show the proximity of several TM cells. In this resolution, the outer cell membrane structure can be clearly observed with no additional intracellular membrane structure. In addition, FIGS. 15B-15C also demonstrated the efficacy of CARS and its ability to show the TM cells and that the cell membrane structure is only displayed in the CARS channel and only the collagen fiber extracellular matrix structure is shown in the TPAF imaging channel. FIGS. 15B-15C shows label-free imaging of TM cells using CARS and a collagen extracellular matrix using TPAF. The image is taken using a 60×1.2NA water objective with 3× digital zoom. The CARS signal is shown in red, and the TPAF signal in green. FIG. 15B displays both the CARS and TPAF channels in the image, clearly showing the TM cells in the CARS channel with arrows indicating the TM cells. FIG. 15C displays only the TPAF channel and the TM cells are not observed without the CARS signal.

Both CARS and TPAF are powerful nonlinear label-free optical imaging techniques that are able to produce images around the TM with excellent imaging resolution. CARS and TPAF were able to be simultaneously used to acquire label-free images around the trabecular meshwork of the eye showing both the TM cells and the collagen extracellular meshwork. In one implementation, the CARS laser photon energy difference was set to the CH₂ vibrational frequency, allowing the detection of the various lipid molecules that compose the plasma membrane of living cells. In addition, the excitation photons used in CARS microscopy can be simultaneously absorbed and autofluoresce by the collagen molecules through TPAF. Combining the two techniques, the collagen structures and the TM cells can be readily observed without exogenous labeling.

TPAF and CARS techniques were used to image deeply into the native TM region of the human eye. Images were taken at multiple depths within the tissue, allowing visualization of the tissue in three dimensions. Similar images can be achieved with histological sections or EM ultra-thin sections; however the method described here has the advantage of being performed on unprocessed, unfixed tissue. This tissue is free from the potential distortions of the fine tissue morphology that can occur within the tissue due to infusion of fixatives and treatment with alcohols. We anticipate this new label-free imaging technique can be used to help elucidate the aqueous outflow of the trabecular meshwork and the effects on the TM cells as the conditions of the TM region changes.

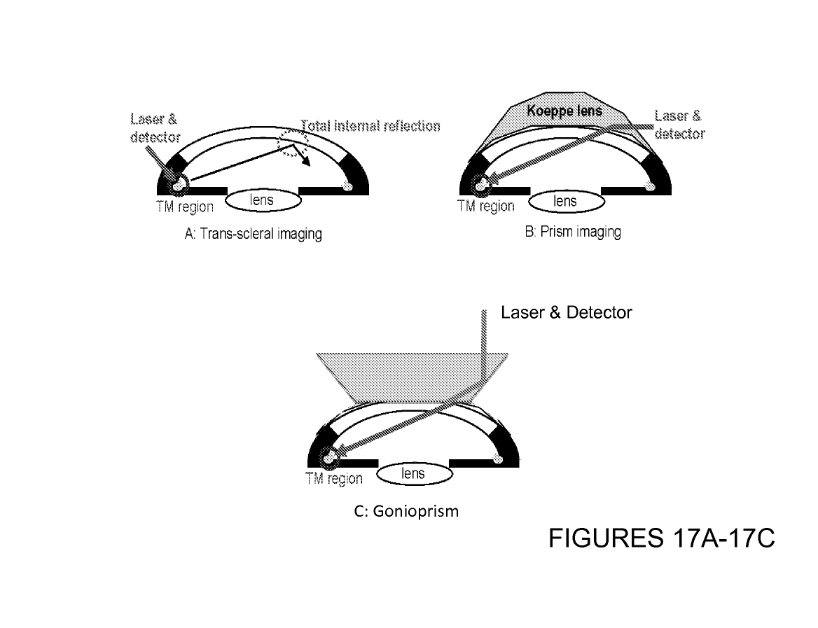

In yet another embodiment, fluorescence lifetime imaging microscopy (FLIM) is used to image tissue, such as the trabecular meshwork (TM) region of the eye. In one example, epithelial cells from the TM region were imaged with a 740 nm two-photon excitation from a Titanium:Sapphire femtosecond laser source. The predominate signal received was from NAD(P)H autofluorescence. The lifetime of each pixel in the image was measured with frequency domain FLIM. This data is plotted on phasor plots which show G(ω) versus S(ω) which are calculated from the amplitude and phase delay of the fluorescence signal. FIGS. 17A-17D show a progression in time of the autofluorescence lifetime in response to addition of a preservative Benzalkonium chloride (BAK). BAK is among the most common preservatives used in ophthalmic preparations for dry eye disease and glaucoma. Clear lifetime changes are shown after a 30 minute time period. The changes indicate a change in the ration of free to protein-bound NAD(P)H which is indicative of cellular response to oxidative stress.

Issues for MPM use in the clinic include accessibility of the different regions of the eye to optical light. For trans-scleral imaging, in general, only the surface of the sclera can be imaged as the highly scattering scleral tissue greatly limits optical light transmission. Others have reported measuring the optical properties of human sclera using an integrating sphere. They found a transmission of 6% at 442 nm, 35% at 804 nm, and 53% at 1064 nm. Although the excitation light for MPM ranging from 800 to 1000 nm can likely penetrate the sclera, the shorter wavelength SHG and autofluorescence emission will be greatly reduced upon collection in the epi-direction.

Another embodiment comprises monitoring drug delivery. For example, it has been reported that two-photon microscopy has been applied to monitor the trans-scleral delivery of tazarotenic acid using its intrinsic fluorescence at 500 nm. The emerging technique of stimulated Raman scattering (SRS) also has great potential for drug delivery monitoring because of the selectivity as well as the linear dependence of the signal on concentration. It has been applied to monitor penetration of dimethyl sulfoxide (DMSO), a skin-penetration enhancer and retinoic acid in the upper dermal layer. There are many opportunities for applying SRS to monitoring drug delivery in the eye due to the transparency of the tissue making deeper penetration depths possible as compared to the skin.

As described above, various embodiments involve imaging of the interior of an eye, such as targeting the trabecular meshwork (TM) region of the eye. FIG. 17A illustrates the difficulty in imaging this region of the eye; fluorescent light emitted from this region does not pass through the cornea, instead it is reflected internally (total internal reflection). In FIGS. 17B and 17C, example routes for imaging a trabecular meshwork of an intact eye are shown. Other routes may be used for imaging other portions of an intact eye, however.

In FIG. 17A, for example, a trans-scleral imaging approach is shown in which the excitation laser beam is directed through a scleral region of the eye to a trabecular meshwork region of the eye. As can be seen in FIG. 17A, sclera tissue (the white part of the eye) extends over the trabecular meshwork of the eye preventing direct viewing of the trabecular meshwork from the front of the eye. In the implementation of FIG. 17A, an excitation beam travels through the scleral region of the eye. The wavelength of the excitation source may be optimized for reduced scattering in the scleral region so that the beam is able to be transmitted through the scleral region and illuminate the trabecular meshwork region of the eye. In one particular implementation, for example, a short pulsed near infrared laser, such as used in two photon microscopy, can penetrate the scleral region of the eye. However, emitted light must be detected through the tissue, unless an additional detector is equipped with a Koeppe lens or Gonioprism (18B and 18C).

In FIG. 17B, a lens or prism, such as a Koeppe lens, is used to direct the excitation beam into the intact eye and illuminate the trabecular meshwork as shown. A Koeppe lens is an ophthalmic prism that can be placed on the eye to nullify the total internal reflection of the cornea. Although a Koeppe lens is shown other types of lenses and/or prisms may be used to illuminate various regions within the intact eye. For example, a specially designed objective lens that has a curvature fitted to a patient's cornea can be developed to bring the laser light to the trabecular meshwork region (or other region) may also be used.

In FIG. 17C, yet another path is shown in which an excitation beam is directed to a trabecular meshwork region of the intact eye using a gonioprism. As described above, the paths shown in FIGS. 17A, 17B, and 17C are merely exemplary. Other paths may be used for imaging portions of an intact eye.

A femtosecond laser excitation source may be used within an imaging system. The femtosecond laser excitation source, can include several types of laser systems with different infra-red wavelengths. The femtosecond laser excitation source, for example, may comprise a femtosecond fiber laser. In various embodiments, for example, the femtosecond fiber laser may comprise a single mode femtosecond fiber laser, a multi-mode fiber laser, a photonic crystal fiber laser, a step index core laser, or a grading index femtosecond fiber laser.

One embodiment, for example, includes (1) a titanium sapphire (TI:Sapphire) laser while another embodiment includes (2) a pulsed fiber laser (e.g., Ytterbium of Erbium gain medium). The excitation source may also serve as a surgical instrument in which a power of a laser source used for the imaging is increased for surgical procedures and/or a separate laser that can be used for surgical purposes based on imaging results of the imaging system. In addition, another excitation light source can be introduced to perform imaging such as spectral optical coherence tomography for alignment purposes. The excitation beams from the femtosecond excitation source are passed through a two-axis scanning mirror stage, a scan lens and a tube lens. The tube lens directs the beams onto a dichroic mirror that separates the excitation beams and emission light received from a sample. The excitation beams are subsequently directed from the dichroic mirror to a microscope objective that in turn focuses the excitation beams onto the sample. The microscope objective may, for example, be mounted on a piezo-electric z-axis scanner for focusing the beams on the sample.

Emission light is received by the objective from the sample and directed back to the dichroic minor that passes the emission light to another dichroic minor that separates the emission light for multimodal acquisition according to the differing wavelengths emitted by the sample. The separated emission light is directed to a filter and a photomultiplier tube for spectral detection of each emission light signal received from the source. The detected spectral data can then be analyzed for imaging.

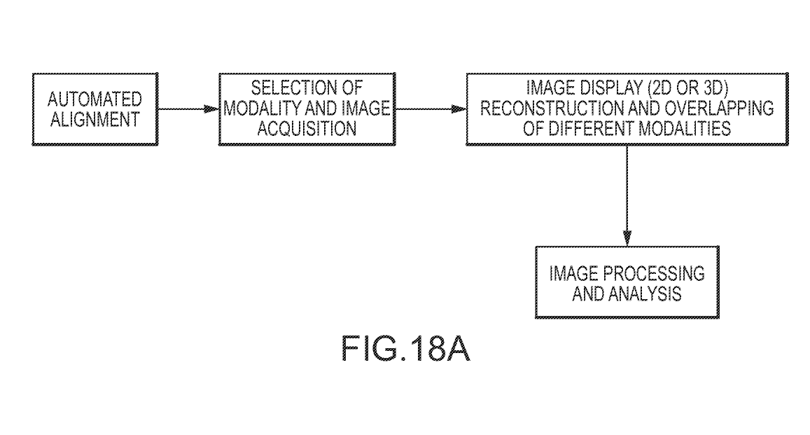

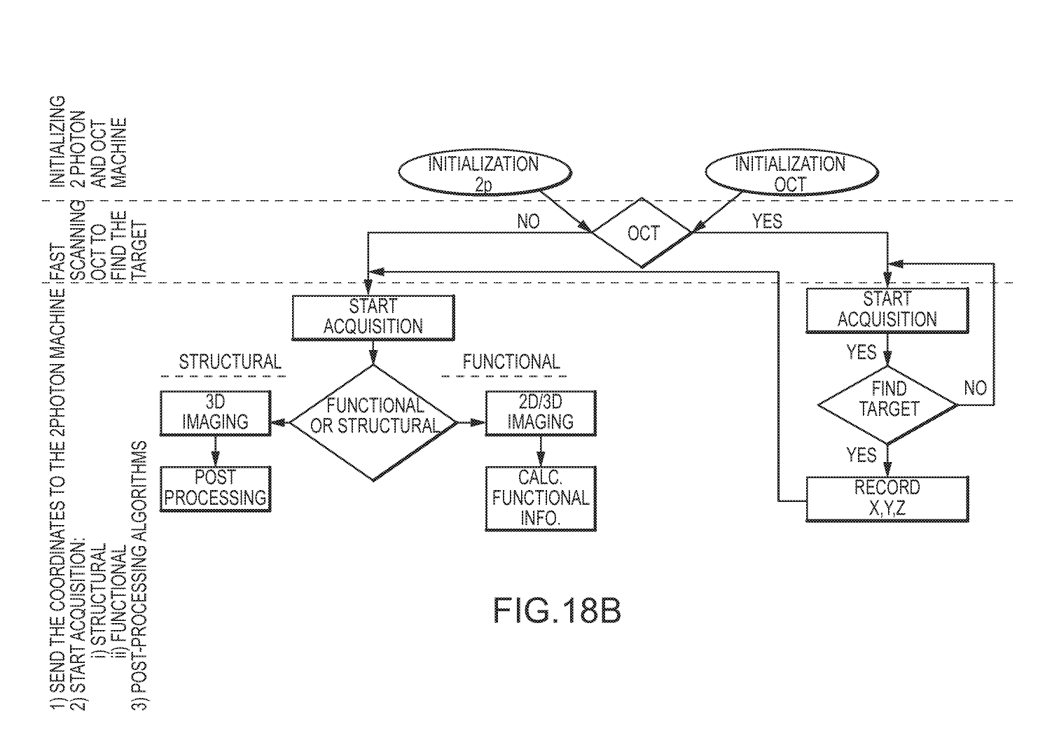

FIG. 18A shows an example process for imaging a portion of an intact eye. Operations of the process may be performed by software and/or hardware modules within an eye imaging system. The process is merely exemplary.

In the process shown in FIG. 18, for example, the imaging apparatus is automatically aligned for imaging a predetermined portion of an intact eye. The alignment, for example, may be accomplished by any method, such as incorporated spectral optical coherence tomography and/or confocal reflectance imaging capabilities.

The process also comprises selecting a modality of image acquisition. Example modalities that may be used to image a portion of an intact eye as described herein include: two photon autofluorescence, autofluorescence fluorescence lifetime, second harmonic generation, third harmonic generation, coherent anti-stokes Raman scattering (CARS), femtosecond CARS, stimulated Raman scattering, stimulated emission microscopy and the like.

The image displayed may be in two or three dimensions. The image is reconstructed by overlapping imaging provided by different modalities used to image the portion of the intact eye.

Image processing and analysis operations are also used to derive information from the multimodal image.

FIG. 18B shows another example process for imaging a portion of an intact eye. Operations of the process may be performed by software and/or hardware modules within an eye imaging system. The process is merely exemplary.

A special designed optical device shaped to the curvature of the cornea (see FIGS. 20 and 21). A custom or non-custom design objective/lens can be used to send the excitation laser source to the TM region. The laser beam can be redirected to the TM region of the eye through total internal reflection between the interface of the optical device and the air, or using an reflective coating such as silver, aluminum, gold or multilayer dielectric coating, to direct the beam into the lens. The optical device can be made out of glass, plastic or other optically transparent material. The optical device can be made out of economic material such as optically transparent plastics, such that the optical device can be disposable. The optical device can be made out of other more expensive material, such as high quality optical glass, that the device can be reused after sterilization. An index-matching fluid can be optionally applied to improve the optical performance between the imaging objective/lens and the optical device, or between the cornea and the optical device. The custom optical device and the objective/lens can be used to image the eye by a single imaging modality or a combination of several other modalities. The objective/lens can be axially or transversely adjustable to image different regions of the eye.

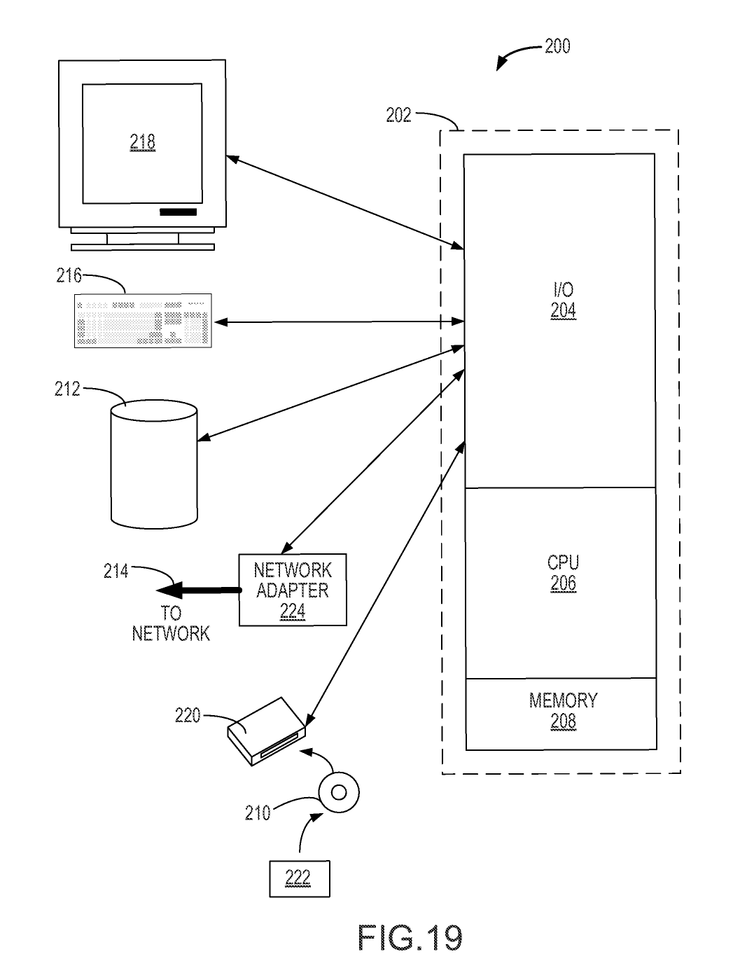

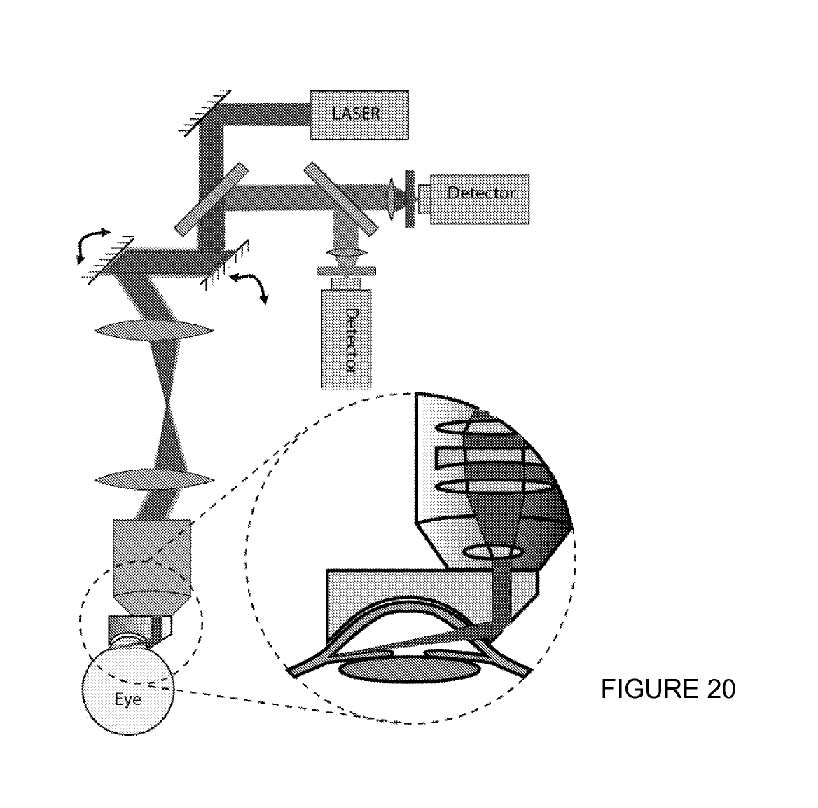

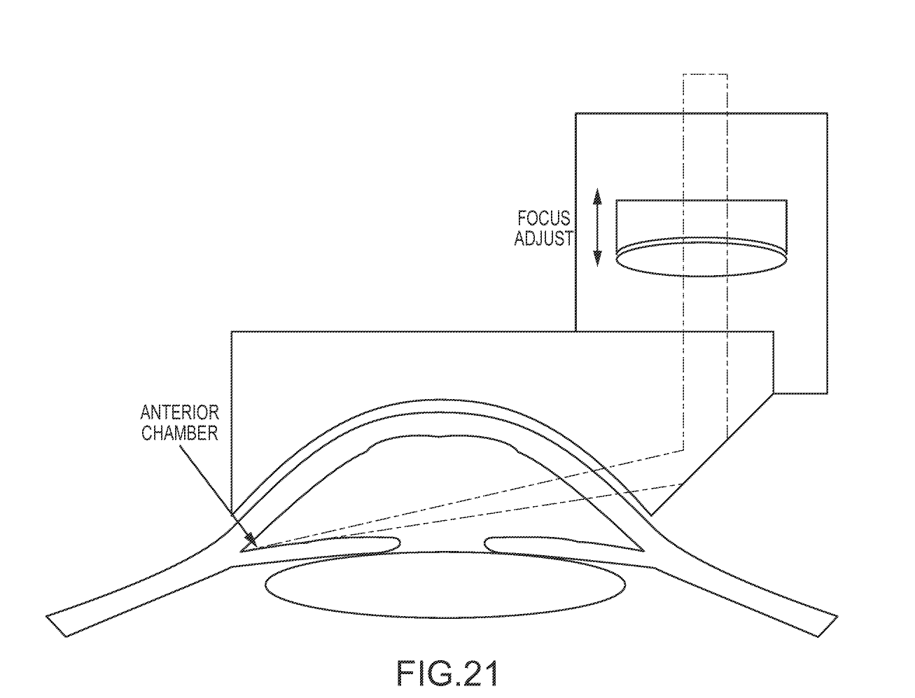

FIG. 19 illustrates an exemplary system useful in implementations of the described technology. A general purpose computer system 200 is capable of executing a computer program product to execute a computer process, such as for alignment, data acquisition and image analysis. Data and program files may be input to the computer system 200, which reads the files and executes the programs therein. Some of the elements of a general purpose computer system 200 are shown in FIG. 19 wherein a processor 202 is shown having an input/output (I/O) section 204, a Central Processing Unit (CPU) 206, and a memory section 208. There may be one or more processors 202, such that the processor 202 of the computer system 200 comprises a single central-processing unit 206, or a plurality of processing units, commonly referred to as a parallel processing environment. The computer system 200 may be a conventional computer, a distributed computer, or any other type of computer. The described technology is optionally implemented in software devices loaded in memory 208, stored on a data storage device (e.g., configured DVD/CD-ROM 210 or other storage unit 212), and/or communicated via a wired or wireless network link 214 on a carrier signal, thereby transforming the computer system 200 in FIG. 19 to a special purpose machine for implementing the described operations.

The I/O section 204 is connected to one or more user-interface devices (e.g., a keyboard 216 and a display unit 218), a disk storage unit 212, and a disk drive unit 220. Generally, in contemporary systems, the disk drive unit 220 is a DVD/CD-ROM drive unit capable of reading the DVD/CD-ROM medium 210, which typically contains programs and data 222. Computer program products containing mechanisms to effectuate the systems and methods in accordance with the described technology may reside in the memory section 204, on a disk storage unit 212, or on the DVD/CD-ROM medium 210 of such a system 200. Alternatively, a disk drive unit 220 may be replaced or supplemented by a floppy drive unit, a tape drive unit, or other storage medium drive unit. The network adapter 224 is capable of connecting the computer system to a network via the network link 214, through which the computer system can receive instructions and data embodied in a carrier wave. Examples of such systems include SPARC systems offered by Sun Microsystems, Inc., personal computers offered by Dell Corporation and by other manufacturers of Intel-compatible personal computers, PowerPC-based computing systems, ARM-based computing systems and other systems running a UNIX-based or other operating system. It should be understood that computing systems may also embody devices such as Personal Digital Assistants (PDAs), mobile phones, gaming consoles, set top boxes, Internet enabled televisions, etc.

When used in a LAN-networking environment, the computer system 200 is connected (by wired connection or wirelessly) to a local network through the network interface or adapter 224, which is one type of communications device. When used in a WAN-networking environment, the computer system 200 typically includes a modem, a network adapter, or any other type of communications device for establishing communications over the wide area network. In a networked environment, program modules depicted relative to the computer system 200 or portions thereof, may be stored in a remote memory storage device. It is appreciated that the network connections shown are exemplary and other devices or means of communications for establishing a communications link between the computers may be used.

In accordance with an implementation, software instructions and data directed toward alignment, data acquisition, and image analysis may reside on disk storage unit, disk drive unit or other storage medium units coupled to the system. The software instructions may also be executed by CPU 206.

The embodiments of the invention described herein are implemented as logical steps in one or more computer systems. The logical operations of the present invention are implemented (1) as a sequence of processor-implemented steps executing in one or more computer systems and (2) as interconnected machine or circuit modules within one or more computer systems. The implementation is a matter of choice, dependent on the performance requirements of the computer system implementing the invention. Accordingly, the logical operations making up the embodiments of the invention described herein are referred to variously as operations, steps, objects, or modules. Furthermore, it should be understood that logical operations may be performed in any order, unless explicitly claimed otherwise or a specific order is inherently necessitated by the claim language.

Although embodiments of this invention have been described above with a certain degree of particularity, those skilled in the art could make numerous alterations to the disclosed embodiments without departing from the spirit or scope of this invention. Specifically, although particular tissues such as eye tissue and other biologic tissues have been described other materials may also be imaged in accordance with the teachings herein. For example, non-biologic structures and industrial imaging of inanimate objects may be imaged in accordance with the teachings herein. Particular example materials are in no way limiting to the applications of any and all imaging techniques discussed herein.

All directional references (e.g., upper, lower, upward, downward, left, right, leftward, rightward, top, bottom, above, below, vertical, horizontal, clockwise, and counterclockwise) are only used for identification purposes to aid the reader's understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the invention as defined in the appended claims.

1. A multimodal method for imaging tissue comprising:
aligning an excitation light source with at least a portion of the tissue;
selecting at least two modalities of image acquisition, the at least two modalities comprising at least two modalities selected from the group consisting of two photon excitation fluorescence, two photon autofluorescence, fluorescence lifetime imaging, autofluorescence lifetime imaging, second harmonic generation, third harmonic generation, coherent anti-stokes Raman scattering (CARS), broadband or multiplex CARS, stimulated Raman scattering, stimulated emission, nonlinear absorption, and micro-Raman microscopy;
imaging the tissue portion with each of the modalities of image acquisition; and
constructing a dual mode image using images from each of the modalities of mage acquisition.

2. The method of claim 1 wherein the tissue comprises eye tissue.

3. The method of claim 2 wherein the eye tissue comprises trabecular meshwork (TM) eye tissue.

4. The method of claim 2 wherein the eye tissue comprises at least one of the group comprising:
cornea, conjunctiva, Schlemm's canal, collector channels, sclera, ciliary body, iris, lens, retina, choroid, optic nerve, vitreous, aqueous humor, blood vessels, and tissues surrounding these structures.

5. The method of claim 1 wherein the aligning operation is performed using incorporated optical coherence tomography.

6. The method of claim 1 wherein the aligning operation is performed using a diode laser based imaging device.

7. The method of claim 1 wherein the aligning operation is performed using confocal reflectance imaging.

8. (canceled)

9. The method of claim 1 wherein the at least two modalities of image acquisition comprise two photon excitation fluorescence or two photon autofluorescence and second harmonic generation.

10. The method of claim 1 wherein the at least two modalities of image acquisition comprise two photon excitation fluorescence or two photon autofluorescence and coherent anti-stokes Raman scattering.

11. The method of claim 1 wherein the tissue comprises an intact eye.

12. The method of claim 11 wherein the portion of the intact eye comprises at least one of the group comprising: trabecular meshwork, Schlemm's canal, collector channels, fluids in the eye, fluids around the eye, tissues in the eye and tissues around the eye.

13. The method of claim 12 wherein the imaging is performed through a scleral tissue of the eye.

14. The method of claim 11 wherein an optical element is used to direct an excitation beam through a cornea of the intact eye.

15. The method of claim 14 wherein the optical element is used to direct an excitation beam through the cornea of the intact eye to an aqueous outflow region of the eye.

16. The method of claim 14 wherein the optical element comprises at least one or more of the group comprising: a lens, a prism, a lens and/or a mirror, a lens and/or an index matching media, a lens and/or an index matching gel media.

17. The method of claim 14 wherein the optical element comprises a Koeppe lens.

18. The method of claim 14 wherein the optical element comprises a gonioprism.

19. The method of claim 1 wherein the at least two modalities of image acquisition comprise at least one multi-photon modality of image acquisition.

20. The method of claim 1 wherein the at least two modalities of image acquisition comprise at least two multi-photon modalities of image acquisition.

21. A multimodal system for imaging tissue comprising:
an excitation light source or light sources;
an optical and alignment system for directing the excitation beam or beams to a sample and receiving an emission beam from the sample;
at least one detector for receiving the emission beam from the sample;
a spectral filtering or dispersing device for providing at least two imaging modalities at the at least one detector, the at least two imaging modalities comprising at least two modalities selected from the group consisting of two photon excitation fluorescence, two photon autofluorescence, fluorescence lifetime imaging, autofluorescence lifetime imaging, second harmonic generation, third harmonic generation, coherent anti-stokes Raman scattering (CARS), broadband or multiplex CARS, stimulated Raman scattering, stimulated emission, nonlinear absorption, and micro-Raman microscopy; and
a processor for analyzing the detected emission beam and constructing a dual mode image using images from each of the modalities of image acquisition.

22.-24. (canceled)

25. The system of claim 21 wherein the excitation light source comprises a picosecond laser source.

26. The system of claim 21 wherein the excitation light source comprises a femtosecond laser source.

27. The system of claim 26 wherein the excitation light source comprises a femtosecond fiber laser source.

28. The system of claim 26 wherein the femtosecond fiber laser source comprises at least one of the group comprising: a single mode femtosecond fiber laser, a multi mode femtosecond fiber laser, a photonic crystal femtosecond fiber laser, a step index core femtosecond fiber laser, and a grading index femtosecond fiber laser.

29. The system of claim 21 wherein the optical and alignment system comprises a non-descanned optical system.

30. The system of claim 21 wherein the optical and alignment system comprises a descanned optical system.

31. The system of claim 21 wherein the optical and alignment system comprises an optical coherence tomography (OCT) imaging system.

32. The system of claim 31 wherein the OCT imaging system comprises at least one of the group comprising: frequency domain, Fourier domain, sweeped source, time domain, polarization sensitive, cross polarized and spectral optical coherence tomography.

33.-45. (canceled)

46. A computer-readable medium storing instructions for performing operations on a computer, the instructions comprising:
instructions for aligning an excitation light source with at least a portion of the tissue;
instructions for selecting at least two modalities of image acquisition, the at least two modalities comprising at least two modalities selected from the group consisting of two photon excitation fluorescence, two photon autofluorescence, fluorescence lifetime imaging, autofluorescence lifetime imaging, second harmonic generation, third harmonic generation, coherent anti-stokes Raman scattering (CARS), broadband or multiplex CARS, stimulated Raman scattering, stimulated emission, nonlinear absorption, and micro-Raman microscopy;
instructions for imaging the tissue portion with each of the modalities of image acquisition; and
instructions for constructing a dual mode image using images from each of the modalities of image acquisition.

47.-64. (canceled)

65. The method of claim 1 wherein the tissue comprises at least one of the group comprising skin tissue, oral tissue, and nasal cavity tissue.

66. The method of claim 1 wherein the tissue comprises a biologic tissue.

67. The method of claim 1 wherein the tissue comprises a non-biologic tissue.

68. The method of claim 1 wherein the tissue comprises industrial imaging of inanimate objects.

69.-72. (canceled)

73. The system of claim 21 wherein the optical and alignment system comprises a coupling agent to allow for overcoming total internal reflection of an imaged tissue.
